(19) United States
(12) Patent Application Publication
(10) Pub. No.: US 2005/0131219 A1
(43) Pub. Date: Jun. 16, 2005

(54) Methods for reducing complexity of a sample using small epitope antibodies

(76) Inventors: Michael S. Urdea, Alamo, CA (US); Gregory M. Landes, Livermore, CA (US); Gregory T. Went, Mill Valley, CA (US)

Correspondence Address: MORRISON & FOERSTER LLP, 755 PAGE MILL RD, PALO ALTO, CA, 94304-1018, US

(21) Appl. No.: 10/921,380

(22) Filed: Aug. 18, 2004

Related U.S. Application Data
(60) Provisional application No. 60/511,720, filed on Oct. 15, 2003.
(60) Provisional application No. 60/496,154, filed on Aug. 18, 2003.

(51) Int. Cl. C12Q 1/68; A23J 1/00; C07K 1/00; C07K 14/00; C07K 16/00; C07K 17/00

(52) U.S. Cl. 530/413

(57) ABSTRACT

The present invention relates generally to methods for reducing the complexity of a sample. More specifically, the present invention relates to proteomics, the measurement of the protein levels in biological samples, and analysis of proteins in a sample using antibodies that recognize small epitopes.

### CROSS-REFERENCE TO RELATED APPLICATIONS

This application claims the benefit of U.S. Provisional Application No. 60/496,154, filed on Aug. 18, 2003, and 60/511,720, filed on Oct. 15, 2003, which are hereby incorporated by reference in their entireties.

### FIELD OF THE INVENTION

The present invention relates generally to methods for reducing the complexity of a sample. More specifically, the present invention relates to proteomics, the measurement of the protein levels in biological samples, and analysis of proteins in a sample using antibodies that recognize small epitopes.

### BACKGROUND OF THE INVENTION

Proteomics offers a more direct look at the biological functions of a cell or organism than does genomics, the traditional focus for evaluation of gene activity. Proteomics involves the qualitative and quantitative measurement of gene activity by detecting and quantitating expression at the protein level, rather than at the messenger RNA level. Proteomics also involves the study of non-genome encoded events including the post-translational modification of proteins, protein degradation and protein byproducts, interactions between proteins, and the location of proteins within the cell. The structure, function, or level of activity of the proteins expressed by a cell are also of interest.

The study of gene expression at the protein level is important because many of the most important cellular processes are regulated by the protein status of the cell, not by the status of gene expression. Also, the protein content of a cell is highly relevant to drug discovery efforts since most drugs are designed to be active against protein targets.

Current technologies for the analysis of protein mixtures, such as the intracellular proteins of a cell or population of cells and the proteins secreted by the cell or population of cells or biological fluids, are based on a variety of protein separation techniques followed by identification and/or analysis of the separated proteins. The most popular method is based on 2D-gel electrophoresis followed by “in-gel” proteolytic digestion and mass spectroscopy. Alternatively, Edman and related methods may be used for the sequencing. This 2D-gel technique requires large sample sizes, is time consuming, and is currently limited in its ability to reproducibly resolve a significant fraction of the proteins expressed by a human cell. Techniques involving some large-format 2D-gels can produce gels which separate a larger number of proteins than traditional 2D-gel techniques, but reproducibility is still poor and over 95% of the spots cannot be sequenced due to limitations with respect to sensitivity of the available sequencing techniques. The electrophoretic techniques are also plagued by a bias towards proteins of high abundance.

Thus, there is a need for the ability to assay more completely proteins expressed by a cell or a population of cells in an organism or in a fluid comprising protein (such as serum, plasma, lymph, and other biological fluids), including up to the total set of proteins expressed by the cell or cells or found in the fluid comprising protein.

### BRIEF SUMMARY OF THE INVENTION

In one aspect, the invention provides methods for reducing the complexity of a sample, said methods comprising: (a) contacting a sample with one or more small epitope antibody under conditions that permit binding; and (b) separating an antibody-protein complex, whereby proteins comprising one or more epitope(s) bound by the one or more small epitope antibody are isolated, separated, enriched and/or purified.

In another aspect, the invention provides methods comprising (a) contacting a sample with one or more small epitope antibody under conditions that permit binding; (b) separating an antibody-protein complex, whereby proteins comprising one or more epitope(s) bound by the one or more small epitope antibody are isolated, separated, enriched and/or purified; and (c) separating proteins from the antibody-protein complex.

In another aspect, the invention provides methods for reducing the complexity of a sample, said methods comprising: separating a small epitope antibody-protein complex, whereby proteins comprising an epitope bound by the small epitope antibody are enriched; wherein the complex was generated by contacting a sample with the small epitope antibody.

In another aspect, the invention provides methods for reducing the complexity of a sample, said methods comprising: (a) separating a small epitope antibody-protein complex, whereby proteins comprising an epitope bound by the small epitope antibody are enriched; wherein the complex was generated by contacting a sample with the small epitope antibody; and (b) separating proteins from the antibody-protein complex.

In another aspect, the invention provides methods for reducing the complexity of a sample, said methods comprising separating protein from a small epitope antibody-protein complex, whereby protein comprising an epitope bound by the small epitope antibody is enriched; wherein the small epitope antibody-protein complex is generated by (a) contacting a sample with the small epitope antibody under conditions that permit binding, whereby the small epitope antibody-protein complex is generated; and (b) separating an antibody-protein complex.

As is evident, one or more steps may be combined and/or performed sequentially (often in any order, as long as the requisite product(s) are able to be formed), and, as is evident, the invention includes various combinations of the steps described herein. It is also evident, and is described herein, that the invention encompasses methods in which the initial, or first, step is any of the steps described herein. Methods of the invention encompass embodiments in which later, “downstream” steps are an initial step.

In some embodiments, the methods further comprise a step of treating the sample with a protein cleaving agent, whereby polypeptide fragments are generated. In embodiments involving a step of separating protein from the antibody-protein complex, the sample can be treated with a protein cleaving agent prior to a step of contacting a sample with the at least one small epitope antibody, and/or following a step of separating protein from the antibody-protein complex. Methods for treatment with protein cleaving agents are well known in the art and described herein. One or more protein cleaving agent may be used. The protein cleaving agent may be an enzyme (such as chymotrypsin or trypsin) or a chemical agent (such as cyanogen bromide).

Thus, in another aspect, the invention provides methods for reducing the complexity of a sample, said methods comprising (a) contacting a sample with one or more small epitope antibody under conditions that permit binding; (b) separating an antibody-protein complex, whereby proteins comprising one or more epitope(s) bound by the one or more small epitope antibody are enriched; (c) separating protein from protein-antibody complex; and (d) treating the protein with a protein cleaving agent, whereby polypeptide fragments are generated.

In another aspect, the invention provides methods for reducing the complexity of a sample, said methods comprising (a) contacting a sample with one or more small epitope antibody under conditions that permit binding, to form an antibody-protein complex; and (b) treating the antibody-protein complex with a protein cleaving agent to produce polypeptide fragments.

In another aspect, the invention provides methods for reducing the complexity of a protein sample, said methods comprising: (a) treating the sample with a protein cleaving agent, whereby polypeptide fragments are generated; (b) contacting the polypeptide fragments with one or more small epitope antibody under conditions that permit binding, whereby antibody-polypeptide complexes are generated; and (c) separating the antibody-polypeptide complex, whereby polypeptides comprising one or more epitope bound by the one or more small epitope antibody are enriched.

In another aspect, the invention provides methods for reducing the complexity of a sample, said method comprising: (a) incubating a reaction mixture, said reaction mixture comprising: (i) a small epitope antibody; and (ii) a sample, wherein incubating is under conditions permitting binding; and (b) separating an antibody-protein complex, whereby protein is enriched.

In another aspect, the invention provides methods for reducing the complexity of a sample, said method comprising: separating an antibody-protein complex, whereby protein is enriched; wherein the antibody-protein complex is generated by incubating a reaction mixture, said reaction mixture comprising: (a) a small epitope antibody; and (b) a sample, wherein incubating is under conditions permitting binding.

In another aspect, the invention provides methods for reducing the complexity of a sample, said method comprising: (a) incubating a reaction mixture, said reaction mixture comprising: (i) a small epitope antibody; and (ii) a sample, wherein incubating is under conditions permitting binding; (b) separating an antibody-protein complex; and (c) separating protein from the protein-antibody complex, whereby protein is enriched.

In another aspect, the invention provides separating protein from a separated protein-antibody complex, wherein the protein-antibody complex is generated by incubating a reaction mixture, said reaction mixture comprising: (a) a small epitope antibody; and (b) a sample, wherein incubating is under conditions permitting binding; and separation of a protein-antibody complex.

In another aspect, the invention provides a method for reducing the complexity of a sample that comprises a mixture of proteins, comprising separating a small epitope antibody-protein complex, wherein proteins comprising an epitope bound by the small epitope antibody are enriched. In some embodiments, the method further comprises separating protein from the antibody-protein complex. In some embodiments, the small epitope antibody binds an epitope consisting of about 3 to about 5 amino acids. In some embodiments, the sample is contacted with a plurality of small epitope antibodies to form a plurality of small epitope antibody-protein complexes. In some embodiments, the small epitope antibodies are detectably labeled. In some embodiments, a plurality of small epitope antibodies is immobilized on a solid matrix. In some embodiments, the sample is contacted with a plurality of small epitope antibodies in parallel. In some embodiments, the sample is contacted with a plurality of small epitope antibodies serially. In some embodiments, the sample is contacted with at least 100 small epitope antibodies. In some embodiments, the method further comprises contacting protein separated from the antibody-protein complex with a protein cleaving agent to form polypeptide fragments. In some embodiments, the method further comprises contacting the small epitope antibody-protein complex with a protein cleaving agent to form polypeptide fragments. In some embodiments, the method further comprises contacting the sample with a protein cleaving agent to form polypeptide fragments prior to formation of the small epitope antibody-protein complex, optionally further comprising separating polypeptide fragments from the small epitope antibody-protein complex. In one embodiment, the protein cleaving agent comprises a protease. In another embodiment, the protein cleaving agent comprises a chemical agent.

In another aspect, the invention provides a method for reducing the complexity of a sample that comprises a mixture of proteins, comprising (a) contacting the sample with at least one small epitope antibody to form an antibody-protein complex; and (b) separating the antibody-protein complex from unbound protein in the sample. In some embodiments, steps (a) and (b) are performed sequentially. In some embodiments, steps (a) and (b) are performed simultaneously. In some embodiments, the method further comprises separating protein from the antibody-protein complex. In some embodiments, the small epitope antibody binds an epitope consisting of about 3 to about 5 amino acids. In some embodiments, the at least one small epitope antibody comprises at least about 100 small epitope antibodies.

In other aspects, the invention provides small epitope antibody-protein complexes, proteins, and/or polypeptide fragments prepared using methods for reducing the complexity of a sample described herein.

As is evident to one skilled in the art, aspects that refer to combining and incubating the resultant mixture also encompass method embodiments which comprise incubating the various mixtures (in various combinations and/or subcombinations) so that the desired products are formed.

One, or more than one (such as about two, about three, about four, about five, about ten, about twenty or more) small epitope antibod(ies) may be used in the methods of the invention. In some embodiments, the sample is contacted with about 20, about 30, about 40, about 50, about 75, about 100, about 125, about 150, about 200, about 300, about 400, about 500, about 1000, or more small epitope antibodies. In some embodiments, the sample is contacted with at least about 20, about 30, about 40, about 50, about 75, about 100, about 125, about 150, about 200, about 300, about 400, about 500, about 1000, or more small epitope antibodies. In some embodiments, the sample is contacted with less than about 100, about 95, about 90, about 85, about 80, about 75, about 70, about 65, about 60, about 55, about 50, about 45, about 40, about 35, about 30, about 25, about 20, about 15, about 10, or fewer small epitope antibodies. In some embodiments, the sample is contacted with at least about any of 10, 20, 30, 40, 50, 75, 100, 125, 150, 200, 300, 400 or 500 small epitope antibodies, with an upper limit of about any of 20, 30, 40, 50, 75, 100, 125, 150, 200, 300, 400, 500, or 1000 small epitope antibodies.

The invention also provides methods using the protein prepared using any of the methods described herein, for example, methods for characterizing a protein, methods of expression profiling, methods of identifying proteins; methods for identifying protein degradation products; methods for identifying change in post-translational modification, and methods for determining the mass, the amount and/or identity of protein(s) in a sample. Methods of genotyping (protein mutation detection), identifying splice variants, determining the presence or absence of a protein of interest, expression profiling; methods for identifying protein degradation products; methods for identifying change in post-translational modification, and protein discovery are also encompassed by the methods of the invention.

Thus, in one aspect, the invention provides methods for characterizing a protein comprising: (a) reducing the complexity of a sample using any of the methods described herein, whereby proteins are enriched and/or purified; and (b) analyzing the proteins (interchangeably termed “products”).

In another aspect, the invention provides methods for characterizing protein comprising analyzing protein; wherein the protein was prepared using any of the methods described herein.

In some embodiments, the step of analyzing comprises determining amount of said proteins, whereby the amount of protein(s) prepared, enriched and/or separated is quantified. In some embodiments, the step of analyzing comprises identifying one or more of said proteins. In some embodiments, the identity of the epitope(s) to which the small epitope antibody(ies) bind is used to assist identification of the enriched proteins. In some embodiments, a protein is identified using any one or more of the following characteristics: sequence; mass; m/z ratio (in embodiments involving mass spectrometric analysis), and/or amino acid composition. In other embodiments, the step of analyzing comprises determining the mass of one or more protein(s). In some embodiments, the step of analyzing includes analysis for the detection of any alterations in the protein, as compared to a reference protein which is identical (at least in part) to the protein sequence other than the sequence alteration. Sequence alterations include mutations (such as deletion, substitution, insertion and/or transversion of one or more amino acid), splice variants, degradation products, and change in glycosylation.

In another aspect, the invention provides methods for characterizing a protein using mass spectrometry, comprising: (a) reducing the complexity of a sample using any of the methods described herein, whereby proteins are enriched and/or purified; and (b) analyzing the proteins (interchangeably termed “products”) which are isolated, purified, prepared and/or separated using any of the methods herein, wherein the analyzing is by mass spectrometry.

In another aspect, the invention provides methods for characterizing protein comprising analyzing protein using mass spectrometry; wherein the protein was prepared using any of the methods described herein; wherein the analyzing is by mass spectrometry. In some embodiments, quantity, mass, and/or identity of a protein is determined. In some embodiments, the methods further comprise use of epitope identity information.

In some embodiments, mass spectrometric is matrix assisted laser desorption/ionization (“MALDI”) mass spectrometry; surface-enhanced laser desorption/ionization (“SELDI”); and/or tandem mass spectrometry (e.g., MS/MS, MS/MS/MS, ESI-MS/MS).

In another aspect, the invention provides methods for determining the identity of a protein in a sample using mass spectrometry, said methods comprising: (a) reducing the complexity of a sample using any of the methods described herein, whereby proteins are enriched and/or purified; (b) analyzing the proteins (interchangeably termed “products”), wherein the analyzing is by mass spectrometry; and (c) determining identity of enriched protein. In some embodiments, the methods further comprise use of epitope identity information.

In another aspect, the invention provides methods for determining the identity of a protein in a sample using mass spectrometry, said methods comprising determining the identity of protein using mass spectrometry; wherein the protein is prepared using any of the methods for reducing complexity of a sample described herein. In some embodiments, the methods further comprise use of epitope identity information.

In another aspect, the invention provides methods for protein expression profiling, wherein in the level of expression of one or more proteins is determined, wherein the protein is prepared using any of the methods for reducing complexity of a sample described herein. In some embodiments, the level of expression is determined using mass spectrometry. In some embodiments, the invention provides methods for comparing the amounts of proteins in two or more samples.

In another aspect, the invention provides methods for protein expression profiling, wherein in the identity of one or more proteins is determined, wherein the protein is prepared using any of the methods for reducing complexity of a sample described herein. In some embodiments, protein identity is determined using mass spectrometry. In some embodiments, the methods further comprise use of epitope identity information. In some embodiments, the invention provides methods for comparing the identity of protein(s) in two or more samples.

In another aspect, the invention provides a method for determining the presence or absence of a protein of interest in a sample, wherein the method comprises detecting the protein of interest, of any, in an enriched protein fraction, wherein the enriched protein fraction is prepared using any of the methods for reducing the complexity of a sample described herein, and wherein detection of the protein of interest indicates presence of the protein in the sample. In one embodiment, detection comprises mass spectrometry.

In another aspect, the invention provides a method for determining the amount of a protein of interest in a sample, wherein the method comprises quantifying the amount of the protein of interest in an enriched protein fraction, wherein the enriched protein fraction is prepared using any of the methods for reducing the complexity of a sample described herein. In one embodiment, quantification of the protein of interest comprises mass spectrometry.

In another aspect, the invention provides a method for identifying the protein in a small epitope antibody-protein complex, wherein the small epitope antibody-protein complex is prepared using any of the methods for reducing the complexity of a sample described herein. In one embodiment, the identification comprises mass spectrometry.

In another aspect, the invention provides a method for identification of a biomarker, wherein the method comprises comparing the proteins in two or more enriched protein fractions, wherein each of the two or more enriched protein fractions is prepared from a sample using any of the methods for reducing the complexity of a sample described herein. In some embodiments, the two or more samples comprise samples from at least one individual who has a disease condition and at least one individual who does not have the disease condition, and presence or absence of the biomarker is indicative of the disease condition. In one embodiment, the invention provides a method for determining presence or absence of a disease condition in an individual, comprising determining the level of a biomarker in a sample from the individual, wherein the biomarker is identified as described herein, and wherein the level of the biomarker is indicative of the presence or absence of the disease condition. In some embodiments, the two or more samples comprise samples from at least one individual who has received treatment for a disease condition and at least one individual who has not received treatment for the disease condition, and presence or absence of the biomarker is indicative of efficacy of the treatment. In one embodiment, the invention provides a method for determining efficacy of treatment for a disease condition in an individual, comprising determining the level of a biomarker in a sample from the individual, wherein the biomarker is identified as described herein, and wherein the level of the biomarker is indicative of the efficacy of treatment. In some embodiments, the two or more samples comprise samples from at least one individual who has been exposed to a toxin or pathogen and at least one individual who has not been exposed to the toxin or pathogen, and presence or absence of the biomarker is indicative of exposure of an individual to the toxin or pathogen. In one embodiment, the invention provides a method for determining exposure of an individual to a toxin or pathogen, comprising determining the level of a biomarker in a sample from the individual, wherein the biomarker is identified as described herein, and wherein the level of the biomarker is indicative of exposure to the toxin or pathogen.

In another aspect, the invention provides compositions and kits comprising one or more small epitope antibodies for use in any of the methods of the invention.

In some embodiments, the invention provides a composition comprising a plurality of small epitope antibodies. In some embodiments, the plurality of small epitope antibodies binds epitopes consisting of about 3 to about 5 amino acids. In some embodiments, the small epitope antibodies are detectably labeled. In some embodiments, the plurality of small epitope antibodies comprises at least about 100 small epitope antibodies.

In some embodiments, the invention provides a kit comprising a plurality of small epitope antibodies. In some embodiments, the plurality of small epitope antibodies binds epitopes consisting of about 3 to about 5 amino acids. In some embodiments, the small epitope antibodies are detectably labeled. In some embodiments, the plurality of small epitope antibodies comprises at least about 100 small epitope antibodies.

### BRIEF DESCRIPTION OF THE DRAWINGS

FIG. 1 shows the reaction pattern using mapping polypeptides spanning sequences of immunization polypeptides for group 2 and group 5 mice, respectively.

FIG. 2 shows the results of a secondary screen of positive antibodies in a phage ELISA, as described in Example 2.

FIG. 3 shows an SPR trace of a single chain antibody derived from phage L50P1_{—}15 against peptides 1, 6, 7, 8, and 9, as described in Example 2.

### DETAILED DESCRIPTION OF THE INVENTION

The invention provides methods using one or more antibodies that bind (generally, specifically bind) small epitopes, termed “small epitope antibodies”, to fractionate a protein mixture based on the presence and/or quantity of small epitopes within protein in the protein mixture, whereby protein(s) comprising the small epitope are isolated, separated, prepared, purified and/or enriched. Use of the methods of the invention thereby provides a means for reducing the complexity of a protein mixture, facilitating subsequent use and/or characterization of the enriched protein components of the sample. Insofar as the small epitope bound by the antibody is known, binding by a small epitope antibody provides information relating to amino acid content of protein(s) bound by the small epitope antibody. Small epitope antibodies are further described herein.

As a general overview, the methods comprise: (a) contacting a sample with at least one small epitope antibody under conditions that permit binding; and (b) separating an antibody-protein complex from proteins that are not bound by the small epitope antibody(ies). Generally, proteins comprising one or more epitope bound by the at least one small epitope antibody are isolated, separated, enriched and/or purified. In some embodiments, the methods further comprise: step (c) of separating protein from the antibody-protein complex. In some embodiments, the methods further comprise a step of treating the sample with a protein cleaving agent prior to step (a) of contacting a sample with the at least one small epitope antibody, or, in embodiments involving separation of protein from the antibody-protein complex, after step (c) of separating protein from the antibody-protein complex.

The methods of the invention are useful for fractionating samples comprising protein, which is accomplished by the use of antibodies (termed “small epitope antibodies”) that recognize epitopes that are present in a multiplicity of proteins (such as, for example, an epitope consisting of or consisting essentially of 3 linear amino acids, 4 linear amino acids, or 5 linear amino acids). Small epitope antibodies suitable for use in the methods of the invention are extensively described herein and exemplified in the Examples. By virtue of the specificity of the small epitope antibodies, proteins (e.g., polypeptides) are separated, enriched and/or purified depending on the presence and/or amount of the small epitope within the protein that is recognized by the small epitope antibody(ies) used in the methods of the invention. Methods using the protein prepared using the methods of the invention are further described herein. As is evident, “reducing the complexity of a sample”, as used herein, encompasses isolating, purifying, separating, enriching and/or purifying proteins (e.g., polypeptides) from a sample. Accordingly, the invention provides methods for purifying and/or enriching protein, methods for isolating protein, methods for separating protein, methods for preparing protein for characterization, methods for preparing protein for mass spectrometry analysis, methods for identifying protein (such as one or a group of proteins), methods for discovering new protein, and methods for quantification of protein in a sample.

In one aspect, the invention provides methods for reducing the complexity of a sample, said methods comprising: (a) contacting a sample with one or more small epitope antibody under conditions that permit binding; and (b) separating an antibody-protein complex, whereby proteins comprising one or more epitope(s) bound by the one or more small epitope antibody are isolated, separated, enriched and/or purified.

In another aspect, the invention further provides methods for purifying and/or enriching protein; isolating protein; separating protein, preparing protein for characterization; preparing protein for mass spectrometry analysis; identifying protein (such as one or more protein, or a group of proteins); discovering a new protein; and/or quantification of protein in a sample, wherein said methods comprising: (a) contacting a sample with one or more small epitope antibody under conditions that permit binding; and (b) separating an antibody-protein complex.

In another aspect, the invention also encompasses methods using the protein prepared using any of the methods of the invention, for example, for characterizing a protein, methods of expression profiling, methods of identifying proteins; methods for identifying protein degradation products; methods for identifying change in post-translational modification, and methods for determining the mass, the amount and/or identity of protein(s) in a sample. For example, these methods can be applied in such areas as protein discovery, expression profiling, drug discovery and diagnostics.

In another embodiment, mass spectrometry is used to characterize the protein prepared using any of the methods of the invention. The protein fraction generated using a small epitope antibody is particularly amenable to analysis using mass spectrometry because the number of proteins (including protein variants) is reduced (as compared with the starting sample) by use of the small epitope antibodies described herein. Insofar as the epitope present within the protein is identified, the amino acid sequence or content of the epitope (termed “epitope sequence” or “epitope amino acid content”) provides further information useful for characterizing and identifying the protein. Mass spectrometry methods have been used to quantify and/or identify proteins. In some embodiments, mass spectrometry analysis generates a polypeptide mass map. Using these results, polypeptide mass mapping may permit identification of the corresponding protein. In other embodiments, mass spectrometry analysis is by tandem mass spectrometer, and generates specific sequence information. Use of this information may result in identification of the corresponding protein at the sequence level. In some embodiments, protein is identified using a method comprising MS analysis of protein prepared using any of the methods of the invention, in combination with epitope sequence or amino acid content information.

One or more than one (such as about 2, about 5, about 7, about 10, about 20, about 30, about 50, about 100, or more) small epitope antibodies may be used in the methods of the invention. In some embodiments, the sample is contacted with about 20, about 30, about 40, about 50, about 75, about 100, about 125, about 150, about 200, about 300, about 400, about 500, about 1000, or more small epitope antibodies. In some embodiments, the sample is contacted with at least about 20, about 30, about 40, about 50, about 75, about 100, about 125, about 150, about 200, about 300, about 400, about 500, about 1000, or more small epitope antibodies. In some embodiments, the sample is contacted with less than about 100, about 95, about 90, about 85, about 80, about 75, about 70, about 65, about 60, about 55, about 50, about 45, about 40, about 35, about 30, about 25, about 20, about 15, about 10, or fewer small epitope antibodies. In some embodiments, the sample is contacted with at least about any of 10, 20, 30, 40, 50, 75, 100, 125, 150, 200, 300, 400 or 500 small epitope antibodies, with an upper limit of about any of 20, 30, 40, 50, 75, 100, 125, 150, 200, 300, 400, 500, or 1000 small epitope antibodies.

In another aspect, the invention provides compositions and kits comprising one or more small epitope antibody for use in any of the methods of the invention. In some embodiments, the kits further comprise instructions for any of the methods described herein.

General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); and The Antibodies (M. Zanetti and J. D. Capra, eds., Harwood Academic Publishers, 1995).

Definitions

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv), single chain (ScFv), mutants thereof, fusion proteins comprising an antibody portion, and any other modified configuration of the immunoglobulin molecule that comprises an antigen recognition site of the required specificity. An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

“Fv” is an antibody fragment that contains a complete antigen-recognition and -binding site. In a two-chain Fv species, this region consists of a dimer of one heavy and one light chain variable domain in tight, non-covalent association. In a single-chain Fv species, one heavy and one light chain variable domain can be covalently linked by a flexible polypeptide linker such that the light and heavy chains can associate in a dimeric structure analogous to that in a two-chain Fv species. It is in this configuration that the three CDRs of each variable domain interact to define an antigen-binding specificity on the surface of the VH-VL dimer. However, even a single variable domain (or half of a Fv comprising only 3 CDRs specific for an antigen) has the ability to recognize and bind antigen, although generally at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge regions.

A “monoclonal antibody” refers to a homogeneous antibody population wherein the monoclonal antibody is comprised of amino acids (naturally occurring and non-naturally occurring) that are involved in the selective binding of an antigen. A population of monoclonal antibodies (as opposed to polyclonal antibodies) are highly specific, in the sense that they are directed against a single antigenic site. The term “monoclonal antibody” encompasses not only intact monoclonal antibodies and full-length monoclonal antibodies, but also fragments thereof (such as Fab, Fab′, F(ab′)₂, Fv), single chain (ScFv), mutants thereof, fusion proteins comprising an antibody portion, and any other modified configuration of the immunoglobulin molecule that comprises an antigen recognition site of the required specificity and the ability to bind to an antigen (see definition of antibody). It is not intended to be limited as regards to the source of the antibody or the manner in which it is made (e.g., by hybridoma, phage selection, recombinant expression, transgenic animals, etc.).

The terms “polypeptide”, “oligopeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

An epitope that “specifically binds” or “preferentially binds” (used interchangeably herein) to an antibody is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to an epitope is an antibody that binds this epitope with greater affinity, avidity, more readily, and/or with greater duration than it binds to other epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

A “sample” encompasses a variety of sample types, including those obtained from an individual. The definition encompasses blood and other liquid samples of biological origin, solid tissue samples such as a biopsy specimen or tissue cultures or cells derived therefrom, and the progeny thereof. A sample can be from a microorganism (e.g., bacteria, yeasts, viruses, viroids, molds, fungi) plant, or animal, including mammals such as humans, rodents (such as mice and rats), and monkeys (and other primates). A sample may comprise a single cell or more than a single cell. The definition also includes samples that have been manipulated in any way after their procurement, such as by treatment with reagents, solubilization, or enrichment for certain components, such as proteins or polynucleotides. The term “sample” encompasses a clinical sample, and also includes cells in culture, cell supernatants, cell lysates, serum, plasma, biological fluid, human tissue propagated in animals, and tissue samples. Examples of a sample include blood, plasma, serum, urine, stool, cerebrospinal fluid, synovial fluid, amniotic fluid, saliva, lung lavage, semen, milk, nipple aspirate, prostatic fluid, mucous, and tears.

The “complexity” of a sample means the number of different protein species, including number of different proteins as well as number of different protein variants (including splice variants, polymorphisms, and protein degradation products).

“Detect” refers to identifying (determining) the presence, absence and/or amount of the object or substance to be detected, and as described herein, detection may be qualitative and/or quantitative.

As used herein, the singular form “a”, “an”, and “the” includes plural references unless indicated otherwise. For example, “an” antibody includes one or more antibodies and “a protein” means one or more proteins.

Methods of the Invention

With respect to all methods described herein, reference to a small epitope antibody also includes compositions comprising one or more of these antibodies. These compositions may further comprise suitable excipients, such as pharmaceutically acceptable excipients, as well as buffers and/or components to enhance stability, which are well known in the art.

Methods for Reducing the Complexity of a Sample

The invention provides methods using one or more antibodies that bind (generally, specifically bind) small epitopes, termed “small epitope antibodies”, to fractionate a protein mixture based on the presence or absence or amount of small epitopes within proteins within the protein mixture, whereby a fraction comprising protein(s) comprising and enriched for the small epitope is generated. As used herein, “enriched” refers to an increase in concentration and/or purity of a protein or peptide in comparison with the concentration and/or purity of the protein or peptide in the sample from which it was derived. Use of the methods of the invention thereby provides a means for reducing the complexity of a protein mixture, facilitating subsequent use and/or characterization of the enriched protein components of the sample. Insofar as the amino acid sequence or composition of the small epitope bound by the antibody is known, binding by a small epitope antibody provides information relating to amino acid sequence and/or content of protein(s) bound by the small epitope antibody. As described herein, epitope identity information (i.e., the amino acid content and/or sequence recognized by a small epitope antibody) may be used in combination with other methods of the invention to, e.g., identify proteins. Small epitope antibodies are further described herein.

The invention further provides methods for purifying and/or enriching protein; isolating protein; separating protein, preparing protein for characterization (e.g., subsequent analysis); preparing protein for mass spectrometry analysis; identifying protein; discovering new protein; and/or quantification of protein in a sample.

As a general overview, the methods comprise: (a) contacting a sample with at least one small epitope antibody under conditions that permit binding; and (b) separating an antibody-protein complex. In one embodiment, steps (a) and (b) occur sequentially. In another embodiment, steps (a) and (b) occur simultaneously. Generally, proteins comprising one or more epitope bound by the one or more small epitope antibody are isolated, separated, enriched and/or purified (i.e., removed from the environment of the original sample). In some embodiments, the methods further comprise: step (c) of separating protein from the antibody-protein complex. In some embodiments, the methods further comprise treating the sample with a protein cleaving agent. In one embodiment, the protein cleaving agent is added prior to step (a) of contacting a sample with the at least one small epitope antibody. In another embodiment, the protein cleaving agent is added after step (c) of separating protein from the antibody-protein complex.

The methods of the invention are useful for fractionating samples comprising protein (such as polypeptides), which is accomplished by the use of antibodies (termed “small epitope antibodies”) that recognize epitopes that are present in a multiplicity of proteins (such an epitope consisting of or consisting essentially of 3 linear amino acids, 4 linear amino acids, or 5 linear amino acids). Small epitope antibodies suitable for use in the methods of the invention are extensively described herein and exemplified in the Examples. By virtue of the specificity of the small epitope antibodies, proteins or peptides (e.g., polypeptides) are separated, enriched and/or purified depending on the presence and/or amount of the small epitope within the protein that is recognized by the small epitope antibody(ies) used in the methods of the invention. As is evident, “reducing the complexity of a sample”, as used herein, encompasses isolating, purifying, separating, enriching and/or purifying proteins or peptides (e.g., polypeptides) from a sample (including removing the proteins or peptides from the environment of the sample).

Accordingly, in one aspect, the invention provides methods for reducing the complexity of a sample, said methods comprising: (a) contacting a sample with one or more small epitope antibody under conditions that permit binding; and (b) separating an antibody-protein complex, whereby proteins comprising one or more epitope(s) bound by the one or more small epitope antibody are isolated, separated, enriched and/or purified. In some embodiments, the methods further comprise: step (c) of separating protein from the antibody-protein complex.

In one embodiment, the invention provides methods for reducing the complexity of a protein sample, said methods comprising separating a small epitope antibody-protein complex, whereby proteins comprising an epitope bound by the small epitope antibody are enriched; wherein the complex was generated by contacting a sample with the small epitope antibody. In another embodiment, the invention provides methods for reducing the complexity of a protein sample, said methods comprising separating a plurality of small epitope antibody-protein complexes, whereby proteins comprising epitopes bound by a plurality of small epitope antibodies are enriched, and wherein the complexes were generated by contacting a sample with the plurality of small epitope antibodies.

In another aspect, the invention provides methods for reducing the complexity of a protein sample, said methods comprising separating protein from a small-epitope antibody-protein complex, whereby protein comprising an epitope bound by the small epitope antibody is enriched; wherein the small epitope antibody-protein complex is generated by contacting a sample with the small epitope antibody under conditions that permit binding, whereby the small epitope antibody-protein complex is generated; and separating an antibody-protein complex from unbound proteins in the sample, if any. In one embodiment, the invention provides methods for reducing the complexity of a protein sample, said methods comprising separating a plurality of proteins from small epitope antibody-protein complexes, whereby protein comprising epitopes bound by a plurality of small epitope antibodies is enriched, and wherein the small epitope antibody-protein complexes are generated by contacting a sample with a plurality of small epitope antibodies under conditions that permit binding to proteins in the sample, whereby small epitope antibody-protein complexes are generated, and separating the antibody-protein complexes from unbound proteins in the sample, if any.

As is evident, one or more steps may be combined and/or performed sequentially (often in any order, as long as the requisite product(s) are able to be formed), and, as is evident, the invention includes various combinations of the steps described herein. It is also evident, and is described herein, that the invention encompasses methods in which the initial, or first, step is any of the steps described herein. Methods of the invention encompass embodiments in which later, “downstream” steps are an initial step.

In some embodiments, the methods further comprise a step of treating the sample with a protein cleaving agent, whereby polypeptide fragments are generated. In embodiments involving step (c) of separating protein from the antibody-protein complex, the sample can treated with a protein cleaving agent prior to step (a) of contacting a sample with the at least one small epitope antibody, and/or following step (c) of separating protein from the antibody-protein complex. The protein cleaving agent may be an enzyme (such as chymotrypsin or trypsin) or a chemical agent (such as cyanogen bromide). Protein cleaving agents and methods for treatment with protein cleaving agents are well known in the art and further described herein.

In another aspect, the invention provides methods for reducing the complexity of a sample, said methods comprising (a) contacting a sample with one or more small epitope antibody under conditions that permit binding; (b) separating an antibody-protein complex, whereby proteins comprising one or more epitope(s) bound by the one or more small epitope antibody are enriched; (c) separating protein from protein-antibody complex; and (d) treating the protein with a protein cleaving agent, whereby polypeptide fragments are generated.

In another aspect, the invention provides methods for reducing the complexity of a sample, said methods comprising (a) contacting a sample with one or more small epitope antibody under conditions that permit binding, thereby forming an antibody-protein complex; and (b) treating the antibody-protein complex with a protein cleaving agent to produce polypeptide fragments.

In another aspect, the invention provides methods for reducing the complexity of a protein sample, said methods comprising: (a) treating the sample with a protein cleaving agent, whereby polypeptide fragments are generated; (b) contacting the polypeptide fragments with one or more small epitope antibody under conditions that permit binding, whereby antibody-polypeptide complexes are generated; and (c) separating the antibody-polypeptide complex, whereby polypeptides comprising one or more epitope bound by the one or more small epitope antibody are enriched.

One, or more than one (such as about two, about three, about four, about five, about ten, about twenty, about one hundred, or more) small epitope antibod(ies) may be used in the methods of the invention. In some embodiments, the sample is contacted with about 20, about 30, about 40, about 50, about 75, about 100, about 125, about 150, about 200, about 300, about 400, about 500, about 1000, or more small epitope antibodies. In some embodiments, the sample is contacted with at least about 20, about 30, about 40, about 50, about 75, about 100, about 125, about 150, about 200, about 300, about 400, about 500, about 1000, or more small epitope antibodies. In some embodiments, the sample is contacted with less than about 100, about 95, about 90, about 85, about 80, about 75, about 70, about 65, about 60, about 55, about 50, about 45, about 40, about 35, about 30, about 25, about 20, about 15, about 10, or fewer small epitope antibodies. In some embodiments, the sample is contacted with at least about any of 10, 20, 30, 40, 50, 75, 100, 125, 150, 200, 300, 400, or 500 small epitope antibodies, with an upper limit of about any of 20, 30, 40, 50, 75, 100, 125, 150, 200, 300, 400, 500, or 1000 small epitope antibodies.

It is understood that the sample may also be contacted with other protein binding agents, including antibodies that are not small epitope antibodies, and other protein binding agents. Such agents may be used simultaneously, sequentially, before or after treatment with small epitope antibodies.

In some embodiments, the sample is treated with one or more antibodies that bind to one or more proteins, preferably proteins that are known to be abundant in the sample, prior to or simultaneously with the step of contacting the sample with one or more small epitope antibodies. For example, in a serum sample, pretreatment may comprise antibodies that bind to albumin, immunoglobulin, and/or other abundant proteins. In one embodiment, proteins in the sample are cleaved with a protein cleaving agent prior to contact with the one or more antibodies that bind to one or more known abundant proteins. In another embodiment, proteins in the sample are cleaved with a protein cleaving agent after contact with the one or more antibodies that bind to one or more known proteins, such as abundant proteins. In one embodiment, the bound protein(s) (such as abundant protein(s)) are removed from the sample prior to contact with the one or more small epitope antibodies. In one embodiment, the method comprises “debulking” of a sample by treatment with one or more antibodies that bind to one or more known proteins in the sample, such as abundant protein(s) (optionally followed by removal of bound proteins), cleavage of proteins in the sample with a protein cleaving agent, and contact of cleaved proteins with one or more small epitope antibodies. In another embodiment, the method comprises treatment of the sample with a protein cleaving agent, debulking of the sample by treatment with one or more antibodies that bind to one or more known proteins, such as abundant protein(s) and/or cleaved polypeptide fragments in the sample (optionally followed by removal of the bound protein(s) and/or polypeptide fragments), and contact of the remaining proteins and/or cleaved polypeptide fragments with one or more small epitope antibodies. In another embodiment, the method comprises debulking of the sample by treatment with one or more antibodies that bind to one or more known proteins, such as abundant protein(s) (optionally followed by removal of the bound protein(s)), contacting the sample with at least one small epitope antibody to form an antibody-protein complex, and treatment of the antibody-protein complex with a protein cleaving agent.

It is further understood that the protein components of the sample that remain following treatment with small-epitope antibodies (i.e., the unbound components) may also be suitable for use in the methods of the invention using protein generated using the methods of the invention. Thus, in some embodiments, the methods using the protein generated using the methods of the invention encompass use of this unbound protein fraction.

Methods and conditions for antibody binding and separation of antibody-protein complexes are well known in the art and further described herein. Generally, the sample is partially or wholly denatured when it is contacted with the small epitope antibody(ies), but denaturation is not required in every embodiment. In some embodiment, step (a) of contacting a sample with two or more antibodies is sequential (as when one antibody is contacted with the sample, then removed, another antibody is contacted with the sample and removed, and so on). In other embodiments, step (a) of contacting with two or more antibodies is in parallel, for example, as when a group of antibodies are contacted with the sample simultaneously. In some embodiments, several groups of two or more antibodies are serially contacted with the sample, for example, group 1 is contacted and removed, group 2 is contacted and removed, and so on.

As noted in the definition, and as used herein, “sample” encompasses a variety of sample types, including those obtained from an individual. In some embodiment, the sample comprises blood, plasma, serum, urine, stool, cerebrospinal fluid, synovial fluid, amniotic fluid, saliva, lung lavage, semen, milk, nipple aspirate, prostatic fluid, mucous, and tears. Suitable samples for use in the methods of the invention are described further herein.

Methods Using Proteins Isolated (Enriched) Using the Methods of the Invention

The proteins isolated or enriched using the methods of the invention can be used for a variety of purposes. For purposes of illustration, methods of characterizing proteins using the proteins enriched and/or purified by the methods of the invention, are described. In some embodiments, the proteins are characterized using mass spectrometry, whereby the proteins may be quantified and/or identified. Methods of genotyping (protein mutation detection), identifying splice variants, determining the presence or absence of a protein of interest, expression profiling; methods for identifying protein degradation products; methods for identifying change in post-translational modification, and methods of protein discovery are also described.

For simplicity and convenience, reference is generally made to “protein(s)”. It is understood that reference to protein encompasses “polypeptides” (interchangeably termed “polypeptide fragments”). As is evident from the discussion herein, in some embodiments, a protein cleaving agent is used to generate polypeptide fragments.

Methods of Characterizing a Protein

The invention provides methods for characterizing (for example, detecting (presence or absence) and/or quantifying) a protein of interest (generally, a polypeptide fragment). In some embodiments, use of the methods of the invention generates one or more fractions of the sample, each of which comprises fewer proteins than in the starting sample, facilitating subsequent characterization of the protein comprised in the fraction. In particular, characterization using mass spectrometry is expected to be enhanced, as further described herein.

Thus, the invention provides methods for characterizing a protein comprising: (a) reducing the complexity of a sample using any of the methods described herein, whereby proteins are enriched and/or purified; and (b) analyzing the proteins (interchangeably termed “products”) which are isolated by any one or of the methods described herein.

In another aspect, the invention provides methods for characterizing a protein comprising: analyzing proteins (interchangeably termed “products”), wherein the protein is prepared using any of the methods for reducing complexity of a sample described herein (including: methods for purifying and/or enriching a protein, methods for isolating a protein, methods for separating a protein, methods for preparing a protein fraction for characterization, methods for preparing a protein fraction for mass spectrometry analysis, methods for identifying a protein (such as one or more protein, or a group of proteins), methods for discovering a new protein, and methods for quantification of protein in a sample.)

The step of analyzing can be performed by any method known in the art or described herein. Methods for analyzing proteins are well known in the art, and include: sodium dodecyl sulphate-polyacrylamide gel electrophoresis (“SDS-PAGE”), isoelectric focusing, separated by such techniques as high pressure liquid chromatography, FPLC, thin layer chromatography, affinity chromatography, gel-filtration chromatography, ion exchange chromatography, and other standard biochemical analyses, immunodetection, protein sequencing, analysis with protein arrays, mass spectrometry, and the like. Thus, the invention includes those further analytical and/or quantification methods as applied to any of the products of the methods herein.

In some embodiments, the step of analyzing comprises determining amount of said proteins, whereby the amount of protein(s) prepared, enriched and/or separated is quantified. It is understood that the amount of enriched protein(s) may be determined using quantitative and/or qualitative methods. Determining amount of protein product includes determining whether product is present or absent.

In some embodiments, the step of analyzing comprises identifying one or more of said proteins. Methods for identifying a protein are known in the art, and include: immunodetection, protein sequencing, and the like. In some embodiments, essentially all of the enriched proteins (purified or enriched from a sample) are identified. In some embodiments, the identity of the epitope(s) to which the small epitope antibody(ies) bind is used to assist identification of the enriched proteins. In some embodiments, a protein is identified using any one or more of the following characteristics: sequence; mass; m/z ratio (in embodiments involving mass spectrometric analysis), amino acid composition, and any other method that provide sufficient information to identify a protein. As used herein, “identify” includes identifying known (previously characterized proteins) as well as discovery of previously unknown or uncharacterized proteins (including protein variants such as mutant proteins, differentially modified proteins (e.g., varying carbohydrate content) and splice variants). In some embodiments, a multiplicity, a large multiplicity or a very large multiplicity of proteins are identified. In other embodiments, at least about 2, 3, 4, 5, 10, 20, 30, 40, 50, 60, 70, 80, 90, 100, 500, or 1000 or more proteins are identified.

In other embodiments, the step of analyzing comprises determining the mass of one or more protein(s).

In some embodiments, the step of analyzing includes analysis for the detection of any alterations in the protein, as compared to a reference protein which is identical (at least in part) to the protein sequence other than the sequence alteration. The sequence alterations may be sequence alterations present in the genomic sequence or may be sequence alterations which are not reflected in the genomic DNA sequences, for example, alterations due to post transcriptional alterations, and/or mRNA processing, including splice variants, and/or post-translational modifications, such as variation in amount of glycosylation, and protein degradation or by-products. Sequence alterations include mutations (such as deletion, substitution, insertion and/or transversion of one or more amino acid).

It is understood that the identity (sequence) of the epitope(s) to which the small epitope antibody(ies) may be used in combination with any of the methods described herein to, e.g., identify proteins.

Method of Characterizing a Protein Using Mass Spectrometry

In some embodiments, mass spectrometry (MS) is used to characterize the proteins isolated using the methods of the invention. Generally, in embodiments involving mass spectrometric analysis, the sample will be treated with a protein cleaving agent (whereby polypeptide fragments are generated), but treatment with a cleaving agent is not required in every embodiment. In some embodiments, the sample is treated with a protein cleaving agent prior to contacting the sample with small epitope antibodies. In other embodiments, the sample is treated with a protein cleaving agent following enrichment of a protein fraction by contacting with a small epitope antibody, separation of antibody-protein complex, and separation of protein from the protein-antibody complex. As noted herein, the protein (such as polypeptide fragments) generated using the methods of the invention are particularly amenable to analysis using mass spectrometry because use of the methods of the invention generates fractions of proteins that are less complex than are the starting sample. Insofar as the epitope present within the protein is known, e.g., the cognate epitope recognized by the small epitope antibody used to purify and/or enrich the protein fraction comprising the protein, the amino acid sequence or content of the epitope (termed “epitope sequence” or “epitope content”) provides further information useful for characterizing and identifying the protein.

Methods for mass spectrometric protein analysis are well known in the art and further described herein. Mass spectrometry methods have been used to quantify and/or identify proteins. (See, e.g., Li et al. (2000) Tibtech 18:151-160; Rowley et al. (2000) Methods 20:383-397; and Kuster and Mann (1998) Curr. Opin. Structural Biol. 8: 393-400.) Mass spectrometric techniques have also been developed that permit at least partial de novo sequencing of isolated proteins. Chait et al. (1993) Science 262:89-92; Keough et al. (1999) Proc. Natl. Acad. Sci. USA 96:7131-6; reviewed in Bergman (2000) EXS 88:133-44.

Polypeptide mass mapping provides a polypeptide mass fingerprint of the protein or protein fraction under analysis, based on its amino acid composition. Polypeptide mass mapping can be obtained using, for example, the MALDI-TOF platform, in which matrix-assisted laser desorption/ionization (MALDI) is used to ionize polypeptides of interest while the time of flight distribution of the ionized polypeptides provides mass to charge ratio specifications for each polypeptide which can be used to query protein sequence databases. The polypeptide mass fingerprints yielded comprise the amino acid composition based on mass and charge determination. Using these results, a small set of polypeptide mass matches may provide sufficient information for the identification of the corresponding protein.

In a second method for protein identification by MS, individual polypeptides in the mixture are fragmented to generate sequence information. Polypeptides are ionized by electrospray (ESI) from the liquid phase, and then sprayed into a tandem mass spectrometer that is capable of resolving polypeptides in a mixture, isolating polypeptides of interest and dissociating individual polypeptide species into constituent amino- and carboxy-terminal-containing fragments by predominantly disrupting polypeptide bonds (collision induced dissociation). The resulting mass spectrum is comprised of the parent ion as well as two overlapping mass ladders of ions derived from the amino- and carboxy-terminal containing fragments. Because each member of a ladder differs in mass-to-charge ratio (termed “m/z”) by 1 amino acid from its nearest mass neighbor in the series, a partial primary sequence can be generated and used to query both protein and translated DNA sequence databases. This mass spectrometry platform provides specific sequence information derived from several polypeptides, which is often more useful for protein identification that a list of polypeptide masses that reflect the amino acid composition of the polypeptide (as generated by other platforms, including SELDI-TOF).

Mass spectrometry methods further permit quantification of proteins that are analyzed, as further described below.

Other mass spectrometry methods are well known in the art, and include: matrix assisted laser desorption/ionization (“MALDI”) mass spectrometry; surface-enhanced laser desorption/ionization (“SELDI”); Tandem mass spectrometry (e.g., MS/MS, MS/MS/MS, ESI-MS/MS, etc.). In some embodiments, tandem mass spectrometry is carried out using a laser desorption/ionization mass spectrophotometer that is further coupled to a quadrupole time-of-flight mass spectrometer QqTOF MS (see e.g., Krutchinsky et al., WO 99/38185). Methods such as MALDI-QqTOFMS (Krutchinsky et al., WO 99/38185; Shevchenko et al. (2000) Anal. Chem. 72: 2132-2141), ESI-QqTOF MS (Figeys et al. (1998) Rapid Comm'ns. Mass Spec. 12-1435-144) and chip capillary electrophoresis (chip-CE)-QqTOF MS (Li et al. (2000) Anal. Chem. 72: 599-609) have been described previously. Mass spectrometers and techniques for using them in methods of the invention are well known to those of skill in the art. A person skilled in the art would understand that any of the components of a mass spectrometer (e.g., desorption source, mass analyzer, detect, etc.) can be combined with other suitable components described herein or those known in the art. For additional information regarding mass spectrometers, see, e.g., Principles of Instrumental Analysis, 3rd ed., Skoog, Saunders College Publishing, Philadelphia, 1985; and Kirk-Othmer Encyclopedia of Chemical Technology, 4th ed. Vol. 15 (John Wiley & Sons, New York 1995), pp. 1071-1094.

Data Analysis of Mass Spectra

The mass spectra data obtained using the mass spectrometry analysis can be used to obtain information on the quantity and/or identity of the enriched protein products obtained using the methods of the invention. Data generated by desorption and detection of polypeptides can be analyzed using any suitable means (e.g., visually, by computer, etc). In one embodiment, data is analyzed with the use of a programmable digital computer. The computer contains code that receives as input, data on the strength of the signal at various molecular masses received from a particular addressable location on the substrate. This data can indicate the number of products detected, optionally including the strength of the signal of a peak value and the determined molecular mass for each product detected.

Data analysis can include the steps of determining signal strength (e.g., height of peaks) of a peak value (e.g., of a particular mass-to-charge value or range of values) detected and removing “outliers” (data deviating from a predetermined statistical distribution). The observed peaks can be normalized, a process whereby the height of each peak relative to some reference is calculated. For example, a reference can be background noise generated by instrument and chemicals (e.g., energy absorbing molecule) which is set as zero in the scale. Then the signal strength detected for each polypeptide or other substances can be displayed in the form of relative intensities in the scale desired (e.g., 100). Alternatively, a standard may be admitted with the sample so that a peak from the standard can be used as a reference to calculate relative intensities of the signals observed for each affinity tagged product detected. Software programs such as the Biomarker Wizard program (Ciphergen Biosystems, Inc., Fremont, Calif.) can be used to aid in analyzing mass spectra.

In some embodiments the amounts of one or more proteins present in a sample is determined, in part, by executing an algorithm with a programmable digital computer. The algorithm identifies at least one peak value in a first mass spectrum of a first sample and in a second mass spectrum of a second sample. The algorithm then compares the signal strength of the peak value of the first mass spectrum to the signal strength of the peak value of the second mass spectrum. The relative signal strengths are an indication of the amount of the protein that is present in the first and second samples. A standard containing a known amount of a protein can be analyzed as the second sample to better quantify the amount of the protein present in the first sample. In certain embodiments, the identities of the proteins in the first and second samples can also be determined (see below).

The present invention also provides methods of determining the identity of a protein. In certain embodiments, a programmable digital computer is used to access a database containing one or more mass spectra. An algorithm is then executed with a programmable digital computer to determine at least a first measure for each of the predicted mass spectra. The first measure is an indication of the closeness-of-fit between a mass spectrum of the protein and each of the plurality of predicted mass spectra.

The data of a mass spectrum can be used to identify the proteins by executing an algorithm with a programmable digital computer that compares the MS data to records in a database. Each molecule provides characteristic mass-spectrometric (MS) data (also referred to as a mass spectral “signature” or “fingerprint”) when analyzed by MS methods. This data can be analyzed by comparing it to databases containing, inter alia, actual or theoretical MS data or protein sequence information. Additionally, a protein may be cleaved into fragments for MS analysis. Information obtained from the MS analysis of fragments is also compared to a database to identify proteins (e.g., proteins) in the sample (see e.g., Yates (1998) J. Mass Spec. 33:1-19; Yates et al., U.S. Pat. No. 5,538,897; Yates et al., U.S. Pat. No. 6,017,693; PCT Publication No. WO 00/11208 and Gygi et al. (1999) Nat. Biotechnol. 10:994-999). Software resources that facilitate interpretation of mass spectra, especially protein mass spectra, and mining of public domain sequence databases are now readily accessible on the Internet to facilitate protein identification. Among these are Protein Prospector (http://prospector.ucsf/edu), PROWL (http://prowl.rockefeller.edu), and the Mascot Search Engine (Matrix Science Ltd., London, UK, www.matrixscience.com).

In certain embodiments, MS data and information obtained from that data are compared to a database consisting of data and information relating to proteins. For example, the database may consist of sequences of nucleotides or amino acids. The database may consist of nucleotide or amino acid sequences of expressed sequence tags (ESTs). Alternatively, the database may consist of sequences of genes at the nucleotide or amino acid level. The database can include, without limitation, a collection of nucleotide sequences, amino acid sequences, or translations of nucleotide sequences included in the genome of any species.

A database of information relating to proteins, e.g., sequences of nucleotides or amino acids, is typically analyzed via a computer program or a search algorithm which is optionally performed by a computer. Information from sequence databases is searched for best matches with data and information obtained from the methods of the present invention (see e.g., Yates (1998) J. Mass Spec. 33: 1-19; Yates et al., U.S. Pat. No. 5,538,897; Yates et al., U.S. Pat. No. 6,017,693). Any appropriate algorithm or computer program useful for searching a database can be used. Search algorithms and databases are constantly updated, and such updated versions will be used in accordance with the present invention. Examples of programs or databases can be found on the World Wide Web (WWW) at http://base-peak.wiley.com/, http://mac-mann6.embl-heidelberg.de/MassSpec/Software.html, http://www.mann.embl-heidelberg.de/Services/PeptideSearch/PeptideSearchln-tro.html, ftp://ftp.ebi.ac.uk/pub/databases/, and http://donatello.ucsf.ed-u. U.S. Pat. Nos. 5,632,041; 5,964,860; 5,706,498; and 5,701,256 also describe algorithms or methods for sequence comparison. Other examples of databases include the Genpept database, the GenBank database (described in Burks et al. (1990) Methods in Enzymology 183: 3-22, EMBL data library (described in Kahn et al. (1990) Methods in Enzymology 183:23-31, the Protein Sequence Database (described in Barker et al. (1990) Methods in Enzymology 183:31-49, SWISS-PROT (described in Bairoch et al. (1993) Nucleic Acids Res. 21: 3093-3096, and PIR-International (described in (1993) Protein Seg. Data Anal. 5:67-192).

In some embodiments, the amino acid sequence of the epitope recognized by the small epitope antibody (termed “epitope sequence”) is used in conjunction with the database search information and search algorithms to enhance identification of proteins. For example, prior to or following analysis of MS data and information obtained from that data by comparison to a database consisting of data and information relating to proteins, the amino acid sequence of the epitope may be used to refine the data analysis. For example, a preliminary list of protein identity candidates may be refined by excluding members from that list that do not include the epitope sequence. In another example, a database may be compiled or theoretically generated of all proteins comprising a given epitope sequence. This database may then be subjected to further analysis using data analysis methods known in the art.

A database of information relating to proteins, e.g., sequences of nucleotides or amino acids, is typically analyzed via a computer program or a search algorithm which is optionally performed by a computer.

In a further embodiment, novel databases are generated for comparison to mass spectrometrically determined MS data, e.g., mass or mass spectra of cleaved protein and polypeptide fragments. For example, a theoretical database of all polypeptide fragments comprising an epitope recognized by a small epitope antibody is generated. This database may be used in conjunction with any of the data analysis tools and methods described herein.

In some embodiments, the mass of a polypeptide derived from a mass spectrum is used to query a database for those masses of proteins or predicted proteins from nucleic acid sequences that provide the closest fit. In this manner, an unknown protein can be rapidly identified without an amino acid sequence. In other embodiments of the invention, the masses provided from polypeptide fragments thereof can be compared to the predicted mass spectra of a database of proteins or predicted proteins from a nucleic acid sequences that provide the closest fit.

Sequences or simulated cleavage fragments from the sequence database that fall within a desired range of similar sequence homologies to sequences generated from the MS data of parent or fragment molecules are designated “matches” or “hits.” In this manner, the identity of the proteins or fragments thereof can be rapidly determined. The investigator can customize or vary the range of acceptable sequence homology comparison values according to each particular analysis.

It is understood that for convenience, reference is made to protein “identity”. It is understood that the methods described herein are equally applicable to the determination of presence or absence of a mutation (such as an amino acid substitution, transversion, insertion or deletion), and other protein variants, such as splice variants, degradation products, and/or differential post-translational modification (for example, variation in glycosylation level).

In some embodiments, the presence or absence of a mutation is determined by detection of a change in m/z ratio relative to a reference m/z ratio.

In some embodiments, level (or changes in level) of post-translational modification is determined by comparing endoglycosylase-treated sample with a reference sample (e.g., a sample that has not been treated with endoglycosylase), whereby level of post translational modification is determined.

Expression Profiling

The methods of the invention are suitable for use in determining the levels of expression of one or more proteins in a sample. As described above, enriched and/or purified protein fractions can be detected and/or quantified by various methods, as described herein and/or known in the art. In some embodiments, protein fractions are analyzed (including quantification and/or identification) using mass spectrometry. It is understood that amount of protein product may be determined using quantitative and/or qualitative methods. Determining amount of product includes determining whether product is present or absent. Thus, an expression profile can includes information about presence or absence of one or more protein sequences of interest. “Absent” or “absence” of product, and “lack of detection of product” as used herein includes insignificant, or de minimus levels.

In some embodiments, the amounts of proteins in two or more samples are compared. Typically, the samples have overlapping protein profiles. Using the methods of the present invention, the amounts of the proteins can be compared to determine how the profiles differ in the nature and amount of proteins that are present. These methods are useful for identifying a change in the nature or amount of a protein that is indicative of a disease state (e.g., a disease biomarker, PSA, BRCA1, etc.) or treatment efficacy, or toxic effects of an agent, or presence of a pathogen (e.g., HIV, bacterial pathogens, viral pathogens, prions, etc), etc. These methods are also useful for discovering proteins that are associated with disease states for drug discovery purposes, diagnostic purposes, etc. In particular, it is useful to compare the protein profiles of samples that are from different subjects or have been subjected to different conditions or treatments.

For example, in certain embodiments, the first sample is an untreated control sample and the second sample has been subjected to an agent or condition. Examples of agents include, but are not limited to: a chemotherapeutic agent, ultraviolet light, a medical device (e.g., a stent defibrillator), an exogenous gene, and a growth factor. Those of skill in the art will recognize that there are many ways to introduce an exogenous gene into a cell (see, e.g., Ausubel et al., eds., (1994), supra). In other embodiments, the first sample is a diseased sample and the second sample is a non-diseased sample. In addition, agents can take the form of candidate drugs. For example, the proteins in a first sample treated with a candidate drug and can be compared to a second sample which is a negative or positive control. The influence of the candidate drug on the amount of a protein (e.g., a protein) present in the first and second sample can be an indication of the candidate drugs efficacy or toxicity. Those of skill in the art will appreciate that these methods can be adapted to analyze the effects of any agent on a disease state or amount of a disease marker present in a sample. In one embodiment, the methods are used to identify protein(s) that are associated with treatment with an agent (such as a candidate drug). Such proteins may be, e.g., may be associated with efficacy of the agent, and thereby serve as a proxy for a clinical endpoint.

Biomarkers

Biomarker protein (or proteins) can be identified using the expression profiling and characterization methods described herein. A biomarker is a protein of interest, for which the detection, monitoring, quantitation, and/or characterization is of interest. In some embodiments, a biomarker is correlated with a specific condition or treatment, such as a disease or condition, treatment with a drug (including efficacy of drug treatment and/or toxicity), treatment with a medical device, and the like. In other embodiments, a biomarker is expressed in a tissue or cell of interest (e.g., a tumor, an organ, etc.). As used herein, a biomarker protein may be a newly identified protein or protein variant (such as a mutant protein, splice variant, a protein with altered post-translational modification, etc.). In other embodiments, a biomarker is a tissue-specific marker.

A biomarker can be used as a surrogate marker in diagnosis (including staging of disease, in some embodiments), prognosis, evaluation and/or selection of therapies, monitoring of disease progression, monitoring of efficacy of treatment, and/or treatment of disease. In some embodiments, a biomarker is detected and/or quantified by any method known in the art, and/or any method described herein, whereby expression of the biomarker (presence or absence of biomarker, or differential expression of the biomarker) indicates the presence of a disorder or a condition. In one embodiment, increase in level of a biomarker indicates the presence of a disorder or condition. In another embodiment, decrease in level of a biomarker indicates the presence of a disorder or condition. In some embodiments, biomarker expression is used to evaluate the efficacy of a particular therapeutic treatment regimen in animal studies, in clinical trials, or to monitor the treatment of an individual subject. In some embodiments, the biomarker serves as a proxy for a desired clinical endpoint. In other embodiments, the biomarker is correlated with efficacy of an agent, as when biomarker expression is predictive of efficacy of treatment with an agent (such as a drug). In one embodiment, increase in level of a biomarker indicates efficacy or progress of treatment. In another embodiment, decrease in level of a biomarker indicates efficacy or progress of treatment.

The biomarker can be used as a marker for toxicity, including, toxicity of an agent such as a pharmaceutical, new drug candidate, cosmetic, or other chemical. In some embodiments, detection of biomarker expression may also be used to monitor for environmental exposure to an agent, such as a toxin or a pathogen. In one embodiment, increase in level of a biomarker indicates toxicity or exposure to an agent. In another embodiment, decrease in level of a biomarker indicates toxicity or exposure to an agent.

A biomarker can be used to screen a plurality or library of molecules and compounds for specific binding affinity, including, for example, DNA molecules, RNA molecules, peptide nucleic acids, polypeptides, mimetics, small molecules, and the like. In one embodiment, an assay involves providing a plurality of molecules and/or compounds, combining a biomarker with the plurality of molecules and/or compounds under conditions to allow specific binding, and detecting specific binding to identify at least one molecule or compound which specifically binds the biomarker.

Similarly, one or more biomarkers, or portions thereof, can be used to screen a plurality or library of molecules and/or compounds in any of a variety of screening assays to identify a ligand. Methods for screening are well known in the art. The assay can be used to screen, for example, aptamers, DNA molecules, RNA molecules, peptide nucleic acids, polypeptides, mimetics, proteins, antibodies, agonists, antagonists, immunoglobulins, inhibitors, small molecules, pharmaceutical agents or drug compounds and the like, which specifically bind the biomarker.

In another embodiment, one or more antibodies comprising an antigen binding site that specifically binds a biomarker can be used for the detection of the biomarker (including in vitro and in vivo detection). In another example, an antibody that specifically binds a biomarker can be linked to an in vivo imaging reagent, such as, for example, ³H, ¹¹¹In, ¹²⁵I, (see Esteban et al. (1987) J. Nucl. Med. 28.861-870), and used in an in vivo imaging application.

Compositions and Kits

The invention also provides compositions for use in any of the methods described herein, such as methods for reducing the complexity of a sample, methods for purifying and/or enriching a protein or a plurality of proteins, methods for isolating and/or separating a protein or a plurality of proteins, and/or methods for preparing a protein, a plurality of proteins, or a protein fraction for characterization, methods for preparing a protein, a plurality of proteins, or a protein fraction for mass spectrometry analysis, methods for identifying a protein or a plurality of proteins, methods for discovering one or more new proteins, methods for detection and/or quantification of a protein or a plurality of proteins in a sample, methods for characterizing a one or more proteins, methods for expression profiling, methods for identifying protein degradation products, methods for identifying change(s) in post-translational modification, and/or methods for determining the mass, the amount and/or identity of protein(s) in a sample. The compositions used in the methods of the invention may comprise one or more (such as about 2, about 3, about 4, about 5, about 7, about 10, about 15 or more) small epitope antibody(ies). In some embodiments, the composition comprises less than about 100, about 95, about 90, about 85, about 80, about 75, about 70, about 65, about 60, about 55, about 50, about 45, about 40, about 35, about 30, about 25, about 20, about 15, about 10, about 5, or fewer small epitope antibodies. In some embodiments, the composition comprises at least about 20, about 30, about 40, about 50, about 75, about 100, about 125, about 150, about 200, about 300, about 400, about 500, about 1000, or more small epitope antibodies. In some embodiments, the composition comprises about 20, about 30, about 40, about 50, about 75, about 100, about 125, about 150, about 200, about 300, about 400, about 500, about 1000, or more small epitope antibodies. In some embodiments, the composition comprises at least about any of 10, 20, 30, 40, 50, 75, 100, 125, 150, 200, 300, 400 or 500 small epitope antibodies, with an upper limit of about any of 20, 30, 40, 50, 75, 100, 125, 150, 200, 300, 400, 500 or 1000 small epitope antibodies.

The invention also provides kits for use in the instant methods. Kits of the invention include one or more containers comprising one or more small epitope antibody(ies). In some embodiments, the kit comprises less than about 100, about 95, about 90, about 85, about 80, about 75, about 70, about 65, about 60, about 55, about 50, about 45, about 40, about 35, about 30, about 25, about 20, about 15, about 10, about 5, or fewer small epitope antibodies. In some embodiments, the kit comprises at least about 20, about 30, about 40, about 50, about 75, about 100, about 125, about 150, about 200, about 300, about 400, about 500, about 1000, or more small epitope antibodies. In some embodiments, the kit comprises about 20, about 30, about 40, about 50, about 75, about 100, about 125, about 150, about 200, about 300, about 400, about 500, about 1000, or more small epitope antibodies. In some embodiments, the kit comprises at least about any of 10, 20, 30, 40, 50, 75, 100, 125, 150, 200, 300, 400 or 500 small epitope antibodies with an upper limit of about any of 20, 30, 40, 50, 75, 100, 125, 150, 200, 300, 400, 500 or 1000 small epitope antibodies. In some embodiments, the kit further comprises instructions for use in accordance with any of the methods of the invention described herein, such as methods for reducing the complexity of a sample, methods for purifying and/or enriching a protein or a plurality of proteins, methods for isolating and/or separating a protein or a plurality of proteins, and/or methods for preparing a protein, a plurality of proteins, or a protein fraction for characterization, methods for preparing a protein, a plurality of proteins, or a protein fraction for mass spectrometry analysis, methods for identifying a protein or a plurality of proteins, methods for discovering one or more new proteins, methods for detection and/or quantification of a protein or a plurality of proteins in a sample, methods for characterizing a one or more proteins, methods for expression profiling, methods for identifying protein degradation products, methods for identifying change(s) in post-translational modification, and/or methods for determining the mass, the amount and/or identity of protein(s) in a sample.

The invention also comprises any of the protein “products” (e.g., proteins enriched, purified, isolated, prepared, separated, and/or fractionated using any of the methods of the invention described herein. The invention also provides proteins or protein fragments characterized (e.g., detected, identified, quantified, etc.) using any of the methods of the invention described herein and compositions comprising such products. Such proteins comprise a cognate small epitope that is recognized by the small epitope antibody (to which the protein was bound). The invention also provides small epitope antibody-protein complexes or small epitope antibody-protein fragment complexes (for methods wherein the proteins are contacted with a protein cleaving agent prior to contact with the small epitope antibody(ies)) prepared or isolated by any of the methods described herein. The invention also provides proteins or protein fragments separated from a small epitope antibody-protein complex or small epitope antibody-protein fragment complex according to any of the methods described herein, and/or protein fragments prepared from proteins after separation from small epitope antibody(ies).

In another aspect, the invention includes compositions and/or kits comprising intermediates (such as complexes, e.g., small epitope antibody-protein complex) produced by any aspect of the methods of the invention. The invention also provides incubation mixtures comprising protein-containing samples and small epitope antibodies and/or small epitope antibody-protein complexes as described herein.

The kits of this invention are in suitable packaging. Suitable packaging includes, but is not limited to, vials, bottles, jars, flexible packaging (e.g., sealed Mylar or plastic bags), and the like. In some embodiments, the kit comprises a container and a label or package insert(s) on or associated with the container. The label or package insert may indicate that the small epitope antibody(ies) are useful for any of the methods described herein, e.g., method for reducing the complexity of a sample, or method for identifying a protein, characterizing a protein, and/or expression profiling. Instructions may be provided for practicing any of the methods described herein.

Components and Reaction Mixtures Useful in the Methods of the Invention

Small Epitope Antibody

The methods of the invention use a small epitope antibody. As used herein, a “small epitope antibody” is an antibody that binds (generally specifically binds) a small peptide epitope. By virtue of the epitope specificity, small epitope antibodies generally recognize a multiplicity of proteins that comprise the small epitope to which the antibody binds. Insofar as the small epitope bound by the antibody is known, binding by a small epitope antibody provides information relating to amino acid content and/or sequence of protein(s) bound by the small epitope antibody. Small epitope antibodies are described, for example, in co-pending U.S. patent application Ser. No. 10/687,174. Small epitope antibodies and methods of making small epitope antibodies are further discussed herein and exemplified in the Examples.

An antibody can encompass monoclonal antibodies, polyclonal antibodies, antibody fragments (e.g., Fab, Fab′, F(ab′)₂, Fv, Fc, etc.), chimeric antibodies, single chain (ScFv), mutants thereof, fusion proteins comprising an antibody portion, and any other modified configuration of the immunoglobulin molecule that comprises an antigen recognition site of the required specificity. The antibodies may be murine, rat, human, or any other origin (including humanized antibodies). Small epitope antibodies may be produced by a number of methods known in the art, including, for example, production by a hybridoma, recombinant production, or chemical synthesis.

Generally, a small epitope antibody binds a short, linear peptide epitope of 3, 4, or 5 sequential (consecutive) amino acids. Alternatively, in some embodiments, a small epitope antibody binds a discontinuous amino acid sequence within a polypeptide. In some embodiments, a small epitope antibody binds an epitope consisting of or consisting essentially of about any of 2, 3, 4, 5, 6, 7, 8, 9, or 10 amino acids. In some embodiments, a small epitope antibody binds an epitope consisting of or consisting essentially of 2 to 10, 3 to 8, or 3 to 5 amino acids. In some embodiments, a small epitope antibody binds an epitope consisting of or consisting essentially of less than about any of 10, 9, 8, 7, 6, 5, 4, or 3 amino acids. In some embodiments, a population of small epitope antibodies binds epitopes consisting of or consisting essentially of about 3 to about 5 amino acids. In some embodiments, a population of small epitope antibodies binds epitopes consisting of or consisting essentially of 2 to 10, 3 to 8, or 3 to 5 amino acids. In some embodiments, a population of small epitope antibodies binds epitopes consisting of or consisting essentially of about any of 2, 3, 4, 5, 6, 7, 8, 9, or 10 amino acids. In some embodiments, a population of small epitope antibodies binds epitopes consisting of or consisting essentially of less than about any of 10, 9, 8, 7, 6, 5, 4, or 3 amino acids. A population of small epitope antibodies comprises a plurality of small epitope antibodies. In one embodiment, the plurality of small epitope antibodies binds epitopes of the same number of amino acids. In other embodiments, the plurality of small epitope antibodies binds epitopes of a mixture of different numbers of amino acids. In any of the embodiments described herein, an epitope may be a sequential or discontinuous sequence within a polypeptide, as described below. In some embodiments, one or more small epitope antibody(ies) may be comprised within a mixture of antibodies that comprises antibodies that bind to epitopes larger that the epitopes recognized by the one or more small epitope antibody(ies).

In some embodiments, the small epitope antibody binds an epitope consisting of or consisting essentially of 3 sequential amino acids (termed a 3mer), four sequential amino acids (termed a 4mer), or five sequential amino acids (termed a 5mer). In other embodiments, the small peptide antibody binds a small “discontinuous” or “degenerate” linear peptide sequence, such as the linear peptide sequence YCxC, wherein x represents any of the 20 natural amino acids (a degenerate linear sequence). In other embodiments, the small epitope antibody binds a non-sequential (discontinuous) sequence within a polypeptide based on conformational proximity of amino acids within the polypeptide to fomm the epitope (for example, a conformational epitope formed by proximity of amino acid residues due to secondary structure within a folded polypeptide). In still other embodiments, the small epitope antibody may bind an epitope consisting of an amino acid sequence that is predicted to be antigenic, using methods well known in the art for predicting antigenicity. Antibodies that bind small linear peptide epitopes have been previously described, as shown in Table 2, below. In some embodiments, the same antibody may bind a sequential sequence on one or more proteins and a discontinuous sequence on one or more proteins.

Small epitope antibodies generally recognize a multiplicity of proteins that comprise the small epitope to which the antibody binds. In some embodiments, the small epitope antibody binds to an epitope present one or more times in about any of 0.1%, 0.5%, 1, 2%, 3%, 4%, 5%, 10%, or more of proteins in a sample. In still other embodiments, the small epitope antibody binds to an epitope present one or more times in about 0.1% to 1% of proteins in a sample. In still other embodiments, the small epitope antibody binds to an epitope present one or more times in approximately 1-5% of proteins in a sample. In still other embodiments, the small epitope antibody binds to an epitope present one or more times in about 0.1% to 1% of proteins in a sample, wherein the small antibody epitope binds to a linear peptide epitope consisting of or consisting essentially of 3 amino acids, 4 amino acids or 5 amino acids. In still other embodiments, the small epitope antibody binds to an epitope present one or more times in about 1-5% of proteins in a sample, wherein the small antibody epitope binds to a linear peptide epitope consisting of or consisting essentially of 3 amino acids, 4 amino acids or 5 amino acids. In still other embodiments, the small epitope antibody binds to an epitope present one or more times in about 5-7% or about 5-10% of proteins in a sample, wherein the small antibody epitope binds to a linear peptide epitope consisting or consisting essentially of 3 amino acids, 4 amino acids or 5 amino acids. In some embodiments, a plurality of small epitope antibodies collectively bind to one or more epitopes present one or more times in about any of at least about any of 0.1%, 0.5%, 1, 2%, 3%, 4%, 5%, 10%, or more of proteins in a sample. In some embodiments, a plurality of small epitope antibodies binds to an epitope present one or more times in about any of 0.1 to 1%, 1 to 5%, 5 to 7%, or 5 to 10% of proteins in a sample. Methods for empirically assessing frequency of an epitope in a sample include: assessment using biochemical approaches, such as binding of an antibody followed by analysis using, for example, 2D gels or mass spectrometry, and sequence based analysis, using, for example, amino acid or nucleic acid sequence databases such as GenBank and SwissProt. Suitable databases are further described herein.

In some embodiments, the epitope recognized by a small epitope antibody further comprises a C-terminal amino acid recognized as a cleavage site by an endopeptidase. For example, the epitope could comprise a C-terminal arginine and/or a lysine, which are each recognized by trypsin as a cleavage site. Following endopeptidase digestion of a protein mixture, the amino acid recognized by the endopeptidase is generally found at the C-terminus of the target peptide; accordingly, an epitope encompassing such an amino acid will also be found at the C-terminus of a target polypeptide, which may increase immunogenicity, and increase the binding energy associated with antibody-target peptide binding.

In some embodiments, the small epitope antibody binds its cognate epitope with an affinity of a binding reaction of at least about 10⁻⁷ M, at least about 10⁻⁸ M, or at least about 10⁻⁹ M, or lower. Binding affinity may be measured by well-known methods in the art, including, for example, by surface plasmon resonance (Malmborg and Borrebaeck (1995) J. Immunol. Methods 183(1):7-13; Lofas and Johnsson (1990) J. Chem. Soc. Chem. Commun. 1526. In some embodiments, a binding interaction will discriminate over adventitious binding interactions in the reaction by at least two-fold, at least five-fold, at least 10- to at least 100-fold or more.

One, or a population of more than one (such as about two, about three, about four, about five, about ten, about twenty, about one hundred or more) small epitope antibod(ies) may be used in the methods of the invention. Thus, in some embodiments, the methods comprise use of at least one small epitope antibody. In other embodiments, the methods comprise use of at least two small epitope antibodies. In still other embodiments, at least about 5, about 10, about 20, about 30, about 40, about 50, about 60, about 75, about 100, about 125, about 150, about 200 about, 300, about 400, about 500, about 750, about 1000, or more small epitope antibodies are used in the methods of the invention. In some embodiments, the sample is contacted with less than about 100, about 95, about 90, about 85, about 80, about 75, about 70, about 65, about 60, about 55, about 50, about 45, about 40, about 35, about 30, about 25, about 20, about 15, about 10, about 5, or fewer small epitope antibodies. In some embodiments, the sample is contacted with at least about 20, about 30, about 40, about 50, about 75, about 100, about 500, about 1000, or more small epitope antibodies. In some embodiments, a sample is contacted with at least about any of 5, 10, 20, 30, 40, 50, 60, 75, 100, 125, 150, 200, 300, 400, 500, or 750 small epitope antibodies, with an upper limit of about any of 10, 20, 30, 40, 50, 75, 100, 125, 150, 200, 300, 400, 500, 750, or 1000 small epitope antibodies. It is understood that mixture of small epitope antibodies and other protein binding agents (such as antibodies that are not small epitope antibodies) may be used.

It is understood that the identity (sequence) of the epitope(s) to which the small epitope antibody(ies) may be used in combination with any of the methods described herein to, e.g., identify proteins. In some embodiments, the small epitope identity is known. In other embodiments, the identity of the epitope is predictable using methods known in the art.

As discussed herein, antibodies may be contacted with the sample one at a time or in groups of two or more antibodies. In some embodiments, contacting is serial (sequential or iterative), e.g., a single antibody or group of antibodies is contacted with the sample and separated, and a second antibody or group of antibodies is contacted with the sample and separated. In other embodiments, contacting is in parallel, e.g., a group of antibodies is contacted with the sample and separated. It is appreciated that contacting may be both in parallel and serial, as when different groups of antibodies are serially contacted with a sample. Groups of antibodies may be overlapping in composition (e.g., group 1=antibody A, B, C, D,; group 2=antibody B, C, D, E, etc.).

It is evident that the number of small epitope antibodies that are useful in the methods of reducing complexity of a sample depends on the use, application, and/or subsequent analysis contemplated for the protein prepared using one or more small epitope antibodies. In some applications, such as detection of a protein(s) comprising a cognate epitope recognized by a small antibody, a single small epitope antibody (or, in some embodiments, a small number of small epitope antibodies) may be used to prepare, purify and/or enrich a fraction of protein(s) that comprises the protein for which subsequent detection (or other analysis) is desired. Then, the separated protein can be subjected to further analysis. In other embodiments, use of a set of two or more small epitope antibodies may be useful. For example, in applications such as protein discovery and, in some embodiments, expression profiling, it may be desirable to use a multiplicity of small epitope antibodies, such that a large multiplicity of proteins (such as essentially all protein in the starting sample) will be enriched and/or purified. Use of a multiplicity of small epitope antibodies is also useful in application in which purification and/or enrichment of new protein(s) or protein forms is desired (for example, because information regarding target protein sequence is unknown). As an illustrative example relating to embodiments involving fractionation of a multiplicity of proteins in a sample (such as essentially all proteins in a sample) shown, knowledge of the sequence and/or the length of the cognate amino acid epitope recognized by the small epitope antibody permits an estimate regarding the expected frequency of the epitope(s) recognized by the small epitope antibody(ies) within the protein sample. As shown in Table 1, there are a total of 8,000 (20³), 160,000 (20⁴) and 3,200,000 (20⁵) random combinations for 3mer, 4mer and 5mer linear peptide sequences, respectively. Considering 500 amino acids as an average length of protein, the probability that it is detected by a single anti-3mer antibody is 0.0625, the probability increases to about 1 when 15 anti-3mer antibodies are used, and the probability increases to 6.25 when 100 anti-3mer antibodies are used. Such calculations are routine. A small epitope antibody may also recognize a degenerate linear epitope, for example a short peptide, such as YCxC, where x represents two or more of the 20 standard amino acids.

TABLE 1
Distribution properties of short linear
amino acid peptides

| | Epitope amino acid length (n) | | | |
|---|---|---|---|---|
| | 2 | 3 | 4 | 5 |
| # of random combinations (20ⁿ) | 400 | 8,000 | 160.000 | 3,200,000 |
| Appearance rate in a 500 mer | 1.25 | 0.0625 | 0.003125 | 0.00015625 |
| protein (500/20ⁿ) | | | | |
| Detection rate by 100 anti-nmer | 125 | 6.25 | 0.3125 | 0.015625 |
| antibodies (100 × 500/20ⁿ) | | | | |
| Detection rate by 1000 anti-nmer | 1,250 | 62.5 | 3.125 | 0.15625 |
| antibodies (1000 × 500/20ⁿ) | | | | |

Thus, it is understood that the number of small epitope antibodies useful in the methods of the invention depends on various factors, including, for example, the use, application, and/or subsequent analysis contemplated for the protein fraction bound by the small epitope antibody(ies), complexity of the sample (in terms of number of expected or estimated or previously determined proteins, including protein variants such as splice variants), average size of the proteins in the sample, frequency that the cognate epitope is present or predicted to be present in a sample, binding affinity and/or specificity of the small epitope antibody(ies); knowledge of target protein(s), and stability of the small epitope antibody. Such factors are well known in the art and are further discussed herein.

Antibodies that bind small linear peptide epitopes have been previously described, as shown in Table 2.

TABLE 2
Published short antibody epitope sequences

| Epitope Seq | Source protein | Antibody | Reference |
|---|---|---|---|
| NKS | Opa of N. meningitidis | U623, U506 | Malorny, B., et al. |
| | | | (1998) J Bacteriol |
| | | | 180(5): 1323-30. |
| NRQD | Opa of N. meningitides | O521 | Id. |
| TTFL | Opa of N. meningitides | AB419 | Id. |
| NIP | Opa of N. meningitides | W320/15, W124 | Id. |
| GAT | Opa of N. meningitides | P515 | Id. |
| EQP | MB of U. urealyticum | 3B1.5 | Zheng, X., et al., |
| | | | (1996) Clin Diagn |
| | | | Lab Immunol 3(6): |
| | | | 774-8. |
| WQDE | Porcine ZP3 beta | mAb-30 | Afzalpurkar, A. et al. |
| | | | (1997) Am J Reprod |
| | | | Immunol 38(1): 26-32. |
| GPGR | Gp120 of HIV-1 | 9x mAbs | Akerblom, L., et al. |
| | | | (1990) Aids 4(10): |
| | | | 953-60. |
| D(A/S)F* | Phosphofructokinase-1 | alpha-F3 | Hollborn, M., et al. |
| | | | (1999) J Mol Recognit |
| | | | 12(1): 33-7. |
| (D/S)GY(A/G)** | Crotoxin | A-56.36 | Demangel, C., et al. |
| | | | (2000) Eur J Biochem |
| | | | 267(8): 2345-53 |

*DAF and DSF.
**Refers to DGYA, DGYG, SGYA and SGYG.

Methods of making small epitope antibodies are known in the art. In another aspect, and as exemplified in the Examples, small epitope antibodies (e.g., human, humanized, mouse, chimeric) may be made by using immunogens which express one or more small peptide epitopes, such as a small linear peptide epitope consisting of or consisting essentially of 3, 4, or 5 amino acids.

Immunogens may be produced, for example, by chemical synthesis. Methods for synthesizing polypeptides are well known in the art. In some embodiments, the polypeptide immunogen is synthesized with a terminal cysteine to facilitate coupling to either KLH or BSA, as is known in the art. The terminal cysteine can be incorporated at the amino terminus of the polypeptide (which may minimize steric effects during immunization and screening), or at the carboxy terminus. In other embodiments, the polypeptide immunogen is synthesized as a multiple antigen polypeptide, or MAP.

The route and schedule of immunization of the host animal are generally in keeping with established and conventional techniques for antibody stimulation and production, as further described herein. General techniques for production of human and mouse antibodies are known in the art and are described herein. Typically, the host animal is inoculated intraperitoneally with an amount of immunogen, including as described herein.

Hybridomas can be prepared from the lymphocytes and immortalized myeloma cells using the general somatic cell hybridization technique of Kohler, B. and Milstein, C. (1975) Nature 256:495-497 or as modified by Buck, D. W. et al., (1982) In Vitro, 18:377-381. Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the small epitope antibodies of the subject invention. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas or progeny cells of the parent hybridomas that produce small epitope antibodies (such as monoclonal antibodies) may be used as source of antibodies or derivatives thereof, or a portion thereof.

Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, can be removed, for example, by running the preparation over adsorbents made of the immunogen attached to a solid phase and eluting or releasing the desired antibodies off the immunogen. Immunization of a host animal with a human or other species of small epitope receptor, or a fragment of the human or other species of small epitope receptor, or a human or other species of small epitope receptor or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glutaradehyde, succinic anhydride, SOCl2, or R1N═C═NR, where R and R1 are different alkyl groups can yield a population of antibodies (e.g., monoclonal antibodies).

If desired, the small epitope antibody (monoclonal or polyclonal) of interest may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity, or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the small epitope and/or greater and/or altered specificity to the small epitope. It will be apparent to one of skill in the art that one or more polynucleotide changes can be made to the small epitope antibody and still maintain its binding ability to the small epitope.

A number of “humanized” antibody molecules comprising an antigen-binding site derived from a non-human immunoglobulin have been described, including chimeric antibodies having rodent or modified rodent V regions and their associated complementarity determining regions (CDRs) fused to human constant domains. See, for example, Winter et al. Nature 349:293-299 (1991), Lobuglio et al. (1989) Proc. Nat. Acad. Sci. USA 86:4220-4224, Shaw et al. (1987) J Immunol. 138:4534-4538, and Brown et al. (1987) Cancer Res. 47:3577-3583. Other references describe rodent CDRs grafted into a human supporting framework region (FR) prior to fusion with an appropriate human antibody constant domain. See, for example, Riechmann et al. (1988) Nature 332:323-327, Verhoeyen et al. Science (1988) 239:1534-1536, and Jones et al. Nature (1986) 321:522-525. Another reference describes rodent CDRs supported by recombinantly veneered rodent framework regions. See, for example, European Patent Publication No. 519,596. These “humanized” molecules are designed to minimize unwanted immunological response toward rodent anti-human antibody molecules which limits the duration and effectiveness of therapeutic applications of those moieties in human recipients. For example, the antibody constant region can be engineered such that it is immunologically inert (e.g., does not trigger complement lysis). See, e.g. PCT/GB99/01441; UK Patent Application No. 9809951.8. There are four general steps to humanize a monoclonal antibody. These are: (1) determining the nucleotide and predicted amino acid sequence of the starting antibody light and heavy variable domains (2) designing the humanized antibody, i.e., deciding which antibody framework region to use during the humanizing process (3) the actual humanizing methodologies/techniques and (4) the transfection and expression of the humanized antibody. See, for example, U.S. Pat. Nos. 4,816,567; 5,807,715; 5,866,692; 6,331,415; 5,530,101; 5,693,761; 5,693,762; 5,585,089; and 6,180,370. Other methods of humanizing antibodies that may also be utilized are disclosed by Daugherty et al., Nucl. Acids Res. (1991) 19:2471-2476 and in U.S. Pat. Nos. 6,180,377; 6,054,297; 5,997,867; 5,866,692; 6,210,671; 6,350,861; and PCT Publication No. WO 01/27160.

In yet another alternative, fully human antibodies may be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse™ from Abgenix, Inc. (Fremont, Calif.) and HuMAb-Mouse® and TC Mouse™ from Medarex, Inc. (Princeton, N.J.).

In an alternative, antibodies may be made recombinantly and expressed using any method known in the art. In another alternative, antibodies may be made recombinantly by phage display technology. See, for example, U.S. Pat. Nos. 5,565,332; 5,580,717; 5,733,743 and 6,265,150; and Winter et al. (1994) Annu. Rev. Immunol. 12:433-455.

Alternatively, the phage display technology (McCafferty et al. (1990) Nature 348:552-553) can be used to produce human antibodies and antibody fragments in vitro, from immunoglobulin variable (V) domain gene repertoires from unimmunized donors. For example, existing antibody phage display libraries may be panned in parallel against a large collection of synthetic polypeptides. According to this technique, antibody V domain genes are cloned in-frame into either a major or minor coat protein gene of a filamentous bacteriophage, such as M 13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for review see, e.g., Johnson, Kevin S. and Chiswell, David J., Current Opinion in Structural Biology (1993) 3, 564-571. Several sources of V-gene segments can be used for phage display. Clackson et al., Nature (1991) 352:624-628 isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Mark et al. (1991) J. Mol. Biol. 222:581-597, or Griffith et al. (1993) EMBO J. 12:725-734. In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling.” Marks, et al. (1992) Bio/Technol. 10:779-783. In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the pM-nM range. A strategy for making very large phage antibody repertoires (also known as “the mother-of-all libraries”) has been described by Waterhouse et al. (1993) Nucl. Acids Res. 21:2265-2266. Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable regions capable of restoring a functional antigen-binding site, i.e., the epitope governs (imprints) the choice of partner. When the process is repeated in order to replace the remaining rodent V domain, a human antibody is obtained (see PCT Publication No. WO 9306213, published Apr. 1, 1993). Unlike traditional humanization of rodent antibodies by CDR grafting, this technique provides completely human antibodies, which have no framework or CDR residues of rodent origin. It is apparent that although the above discussion pertains to humanized antibodies, the general principles discussed are applicable to customizing antibodies for use, for example, in dogs, cats, primates, equines and bovines.

Antibodies may be made recombinantly by first isolating the antibodies made from host animals, obtaining the gene sequence, and using the gene sequence to express the antibody recombinantly in host cells (e.g., CHO cells). Another method that may be employed is to express the antibody sequence in plants (e.g., tobacco), transgenic milk, or in other organisms. Methods for expressing antibodies recombinantly in plants or milk have been disclosed. See, for example, Peeters et al. (2001) Vaccine 19:2756; Lonberg, N. and D. Huszar (1995) Int. Rev. Immunol 13:65; and Pollock et al. (1999) J Immunol Methods 231:147. Methods for making derivatives of antibodies, e.g., humanized, single chain, etc. are known in the art.

Immunoassays and flow cytometry sorting techniques such as fluorescence activated cell sorting (FACS) can also be employed to isolate antibodies that are specific for the desired small epitope.

The antibodies can be bound to many different carriers. Carriers can be active and/or inert. Examples of well-known carriers include polypropylene, polystyrene, polyethylene, dextran, nylon, amylases, glass, natural and modified celluloses, polyacrylamides, agaroses and magnetite. The nature of the carrier can be either soluble or insoluble for purposes of the invention. Those skilled in the art will know of other suitable carriers for binding antibodies, or will be able to ascertain such, using routine experimentation.

DNA encoding small epitope antibodies may be isolated and sequenced, as is known in the art. Generally, the monoclonal antibody is readily isolated and sequenced using conventional procedures (e.g., by using oligonucleotide probes that are capable of binding specifically to genes encoding the heavy and light chains of the monoclonal antibodies). The hybridoma cells serve as a preferred source of such cDNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences, Morrison et al. (1984) Proc. Nat. Acad. Sci. 81: 6851, or by covalently joining to the immunoglobulin coding sequence all or part of the coding sequence for a non-immunoglobulin polypeptide. In that manner, “chimeric” or “hybrid” antibodies are prepared that have the binding specificity of a small epitope antibody (such as a monoclonal antibody) herein.

Small epitope antibodies may be characterized using methods well-known in the art, some of which are described in the Examples. For example, one method is to identify the epitope to which it binds, including solving the crystal structure of an antibody-antigen complex, competition assays, gene fragment expression assays, and synthetic polypeptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence to which a small epitope antibody binds. Epitope mapping is commercially available from various sources, for example, Pepscan Systems (Edelhertweg 15, 8219 PH Lelystad, The Netherlands). Polypeptides of varying lengths (e.g., at least 4-6 amino acids long) can be isolated or synthesized (e.g., recombinantly) and used for binding assays with an anti-small epitope antibody. In another example, the epitope to which the small epitope antibody binds can be determined in a systematic screening by using overlapping polypeptides derived from the small epitope extracellular sequence and determining binding by the small epitope antibody. Certain epitopes can also be identified by using large libraries of random polypeptide sequences displayed on the surface of phage particles (phage libraries), as is well known in the art.

Yet another method which can be used to characterize an anti-small epitope antibody is to use competition assays with other antibodies known to bind to the same antigen, i.e., to determine if the anti-small epitope antibody binds to the same epitope as other antibodies. Competition assays are well known to those of skill in the art.

The small epitope antibodies useful in this invention may be linked to a labeling agent (alternatively termed “label”) such as a fluorescent molecule (such as a hapten or fluorescent bead), a binding partner, a solid support, or other agents to facilitate separation that are known in the art. Such agents are further described herein.

In some embodiments, one or more of the following considerations are used in the design of small epitope antibodies (whether designed to be used singly or in a population) that result in an epitope frequency with sufficient redundancy to yield optimal coverage of the proteins present in a sample. In one embodiment, a group of small epitope antibodies designed according to one or more of the following considerations is capable of binding to cognate epitopes on at least about any of 10, 20, 30, 40, 50, 60, 70, 80, 90, or 95% of the proteins in a sample.
Epitope size: Small epitope antibodies that are small enough to occur frequently in the proteome but large enough to confer sufficient binding energy when recognized by their cognate epitope. In some embodiments, the epitope size recognized by each antibody is 3, 4, or 5 amino acids.
Epitope abundance: In an embodiment, optimal epitope abundance enables each small epitope antibody to bind to about 100 to about 150 serum-derived polypeptides of about 20 to about 100 amino acids in length. This abundance level matches the resolving power of most mass spectrometers without requiring MS-MS and collision-induced dissociation (CID). Epitopes of the appropriate abundance are preferable for achievement of the desired MS resolution and sensitivity.
Sampling redundancy: In some embodiments, a sufficiently large collection of small epitope antibodies is used to permit binding to about 3 to about 5 epitopes per protein per proteome of interest. This design feature provides for sampling redundancy to accommodate the variability expected in both expression levels for different proteins and binding efficiency for each antibody in the collection.
Affinity: In some embodiments, the tightness of binding between small epitope antibodies and their epitopes affects the sensitivity of protein profiling. In some embodiments, each antibody in a collection binds with high enough affinity to ensure that sufficient analyte is captured for MS analysis.
Frequency of Binding: In some embodiments, frequency of binding of small epitope antibodies is high so that peptides present within each bound peptide fraction contain a common epitope. This provides sampling redundancy and facilitates bioinformatic determination of peptide identity.

Contacting the Sample with a Small Epitope Antibody and Separation of Protein from a Protein-Antibody Complex

Methods and conditions for contacting an antibody with a protein in a sample are well known in the art. Antibodies may be contacted with the sample one at a time or in groups of two or more antibodies). In some embodiments, contacting is serial (sequential, or iterative), e.g., a single antibody or group of antibodies is contacted with the sample; separated; and a second antibody or group of antibodies is contacted with the sample, and separated, and so on). In other embodiments, contacting is in parallel, e.g., a group of antibodies is contacted with the sample, and separated. It is appreciated that contacting may be both in parallel and serial, as when different groups of antibodies are serially contacted with a sample. Groups of antibodies may be overlapping in composition (e.g., group 1=antibody A, B, C, D; group 2=antibody B, C, D, E, etc.) or different in composition. Contacting of an antibody with protein may occur with both antibody and protein in a liquid medium or may occur with one component (antibody or protein) bound or associated with a solid support and the other component in a liquid medium. In one embodiment, a liquid (e.g., aqueous) protein containing sample is contacted with a small epitope antibody that is bound or associated with a solid support.

In some embodiments involving parallel contacting, it is desirable for small epitope antibodies to be individually separable, for example, by linking the antibody to detectable distinct beads, use of individually separable binding partners, immobilization of antibody in, e.g., different wells of a multiwell plate, use of antibody arrays, and the like. Insofar as the small epitope bound by the antibody is known, binding by a small epitope antibody provides information relating to amino acid content and/or sequence of protein(s) bound by the small epitope antibody. In embodiments wherein knowledge of the cognate small epitope is desired, it may be convenient to individually separate the small antibodies (such that the protein bound by each small epitope antibody is kept separate). However, individual separation or separability is not required in every embodiment. For example, small epitope antibodies may be combined in small pools of two or more antibodies that possess overlapping antibody composition, such as (1) antibodies ABC; (2) antibodies CDE; (3) antibodies FGH, and (4) antibodies HIJ. Following separation of antibody-protein complexes, and separation of antibody from antibody-protein complexes, information regarding presence or absence of a particular small epitope may be inferred based on membership in a particular group.

To facilitate separation of the antibody-protein complex from unbound protein in the sample, the antibody may be linked to an agent that facilitates separation, such as a binding partner (e.g., biotin, oligonucleotide, aptamer), a solid support (such as a bead or matrix, including a microarray or multiwell plate); or any other agent known in the art. Linking may be covalent or noncovalent, and may be direct or indirect. Methods for linking antibodies to such agents are well known in the art. See, e.g. Kennedy et al. (1976) Clin. Chim. Acta 70:1-31, and Schurs et al. (1977) Clin. Chim. Acta 81:140 (describing coupling techniques, including the glutaraldehyde method, the periodate method, the dimaleimide method, the m-maleimidobenzyl-N-hydroxy-succinimide ester method, all of which methods are incorporated by reference herein).

Methods for separating an antibody-protein complex from a sample are known in the art and include use of a capture agent that binds a binding partner (e.g., avidin to capture a biotin-linked antibody; an oligonucleotide to capture an oligonucleotide linked to an antibody; Physical separation may also be used, such as sedimentation, filtration, FACS (for example, using beads that are labeled with a spectral signature), and magnetic separation (when the antibody is linked to a matrix with magnetic properties, such as a magnetic bead).

Many binding partners are known in the art (e.g., a dinitrophenyl group, digoxigenin, fluorophores, Oregon Green dyes, Alexa Fluor 488 (Molecular Probes), fluorescein, a dansyl group, Marina Blue (Molecular Probes), tetramethylrhodamine, Texas Red (Molecular Probes), BODIPY (4,4-difluoro-4-bora-3a,4a-diaza-s-indacene; U.S. Pat. No. 4,774,339) dyes, etc) that can be used in the present invention. Antibodies that can be used as capture reagents can specifically bind to binding agents are commercially available from vendors such as Molecular Probes, Eugene, Oreg. These antibodies include antibodies that can specifically bind to a dinitrophenyl group, a digoxigenin, a fluorophore, Oregon Green dyes, Alexa Fluor 488 (Molecular Probes), fluorescein, a dansyl group, Marina Blue (Molecular Probes), tetrahmethylrhodamine, Texas Red (Molecular Probes), and a BODIPY dye (Molecular Probes). Any suitable ligand and anti-ligand may also be used.

Oligonucleotides can be used as binding partner and capture reagents. Oligonucleotides include nucleic acids such as DNA, RNA, and mixed RNA/DNA molecules. The oligonucleotide that is used as the affinity label should be able to hybridize to the sequence of the oligonucleotide present on the capture reagent. Those of skill in the art will recognize that many different oligonucleotide sequences can be designed that will hybridize to each other. Important considerations for designing such oligonucleotide pairs include the actual nucleotide sequence, the length of the oligonucleotides, the hybridization conditions (e.g., temperature, salt concentration, presence of organic chemicals, etc.) and the melting temperature of the oligonucleotide.

Solid supports suitable for immobilizing (linking) antibodies or proteins from a sample (and modifications to render solid supports suitable for immobilizing antibodies) are well known in the art. Examples of a solid support include: a bead (including magnetized beads), microwell plate, and a protein microarray (e.g., technology owned by Zyomyx, Inc. See, e.g. U.S. Pat. No. 6,365,418). Thus, for example, CdSe-CdS core-shell nanocrystals enclosed in a silica shell can be easily derivatized for coupling to a biological molecule. Bruchez et al. (1998) Science 281: 2013-2016. Similarly, highly fluorescent quantum dots (zinc sulfide-capped cadmium selenide) have been covalently coupled to biomolecules for use in ultrasensitive biological detection. Warren and Nie (1998) Science 281: 2016-2018. Fluorescently labeled beads are commercially available from Luminex and Quantum Dot.

The bound protein (or in some embodiments, polypeptide fragments) may be released from the antibody-protein complex using conventional immunoaffinity elution conditions such as acidic pH, ionic strength, detergents or combinations of the above. Generally, peptide or protein is de-salted for subsequent fractionation, characterization, or other analysis.

a. Protein Cleaving Agent

In some embodiments, the methods of the invention further comprise treating the sample with a protein cleaving agent, whereby polypeptide fragments are generated. In one embodiment, the sample is contacted with a protein cleaving agent prior to contacting a sample with at least one small epitope antibody. In another embodiment, protein is contacted with a protein cleaving agent after separation of protein from an antibody-protein complex.

Protein cleaving agent treatment generates protein cleavage fragments (such as polypeptides), which can facilitate subsequent mass spectral analysis of the amount of protein and the identity of proteins in a sample(s). In particular, treatment with a protein cleaving agent treatment can facilitate the analysis of proteins whose molecular masses exceed 25 kDa. Protein cleaving reagent treatment also may facilitate accessibility and/or access of small epitope antibodies to a cognate epitope. Protein cleaving agents are well known in the art, and are further discussed herein. In some embodiments, one protein cleaving agent is used. In other embodiments, more than one protein cleaving reagent is used. In some embodiments, more than one type of protein cleaving agent is used with respect to a single sample (e.g., two or more types of proteases, two or more types of chemical cleavage agents, or a combination of one or more protease and one or more chemical cleavage agent). Conditions for treatment with a protein cleaving agent are well known in the art.

In one embodiment, a protein cleaving agent is a protease Example of proteases that can be used as protein cleaving agents, include, but are not limited to: chymotrypsin, trypsin (arg, lys cleavage sequence), thermolysin (phe, leu, iso, val cleavage sequence), V8 protease, Endoproteinase Glu-C, Endoproteinase Asp-N, Endoproteinase Lys-C, Endoproteinase Arg-C, Endoproteinase Arg-N, Factor Xa protease, thrombin, enterokinase, V5 protease, and the tobacco etch virus protease. Proteases useful in the methods of the invention can be genetically engineered and/or chemically modified to prevent autolysis. It is appreciated that an enzymatic protein cleaving agent (such as a protease) can be modified to facilitate removal of the protease from the polypeptide cleavage products following polypeptide cleavage. Such modifications are known in the art and include: (1) bead-bound (e.g., latex, silica or magnetic bead) protease, (2) haptenated protease, (3) affinity depletion of the protease (with, for example, a bead-bound anti-protease, or bead-bound non-cleavable substrate) and/or (4) size exclusion chromatography. The activity of a protease can be inhibited, for example, by treating with heat, a protease inhibitor, a metal chelator (e.g., EGTA, EDTA), etc.

In another embodiment, a protein cleaving agent is a chemical cleaving agent, such as chemical substances and compounds that cleave polypeptides and peptide bonds. Nonlimiting examples of chemical cleaving agents include cyanogen bromide (which cleaves at methionine residues), hydroxylamine (which cleaves between an Asn and a Gly residue), and acid pH (which can cleave an Asp-Pro bond) (see e.g., Ausubel et al., supra).

In still further embodiments, phosphatases (e.g., alkaline phosphatase, acid phosphatase, protein serine phosphatase, protein tyrosine phosphatase, protein threonine phosphatase, etc.), lipases, and other enzymes can be employed as protein cleaving agents.

Sample

As noted in the definition and as used herein, “sample” encompasses a variety of sample types and/or origins, such as blood and other liquid samples of biological origin, solid tissue samples such as a biopsy specimen or tissue cultures or cells derived therefrom, and the progeny thereof. The definition also includes samples that have been manipulated in any way after their procurement, such as by treatment with reagents, solubilization, or enrichment for certain components, such as proteins or polynucleotides. The term “sample” encompasses a clinical sample, and also includes cells in culture, cell supernatants, cell lysates, serum, plasma, biological fluid, and tissue samples. A sample can be from a microorganism, e.g., bacteria, yeasts, viruses, viroids, molds, fungi, plants, animals, including mammals such as humans. A sample may comprise a single cell or more than a single cell. Examples of a sample include blood, plasma, serum, urine, stool, cerebrospinal fluid, synovial fluid, amniotic fluid, saliva, lung lavage, semen, milk, nipple aspirate, prostatic fluid, mucous, cheek swabs, and/or tears.

These samples can be prepared by methods known in the art such as lysing, fractionation, purification, including affinity purification, FACS, laser capture microdissection (LCM) or isopycnic centrifugation. In some embodiments, subcellular fractionation methods are used to create enriched cellular or subcellular fractions, such as subcellular organelles including nuclei, mitochondria, heavy and light membranes and cytoplasm.

Prior to contacting the sample with one or more small epitope antibodies, the sample may be treated with agents capable of denaturing and/or solubilizing proteins, such as detergents (ionic and non-ionic), chaotropes and/or reducing agent. Such agents are known in the art.

Under certain circumstances, it may be desirable to remove or minimize abundant proteins present in a sample, for example, by targeted immunodepletion, or other methods known in the art. Generally, such removal (or reduction) occurs prior to contacting the sample with one or more small epitope antibodies (however, such reduction or removal can occur during or after treatment with small epitope antibodies). Any suitable reagent may be used, including one or more small epitope antibody(ies). In one embodiment, removal and/or reduction of one or more sample components is effected by treating the sample with one or more small epitope antibodies.

In some embodiments, it may be desirable to treat the sample with a polysaccharide cleaving agent, for example, to reduce, minimize, and or eliminate glycosylation of sample protein. Removal of any carbohydrate moieties may be accomplished chemically or enzymatically. Examples of polysaccharide cleaving agents include glycosidases, endoglycosidases, exoglycosylases, and chemicals such as trifluoromethanesulfonic acid. Endoglycosidases such as Endoglycosidase H (New England Biolabs, Beverly, Mass.), and Endo H_{f} (New England Biolabs) are commercially available. These endoglycosidases cleave the chitobiose core of high mannose and some hybrid oligosaccharides from N-linked glycoproteins. Exoglycosidases are also commercially available from vendors such as New England Biolabs and include, beta-N-Acetylhexosaminidase, alpha-1-2-Fucosidase, alpha-1-3,4 Fucosidase alpha-1-2,3 Mannosidase, alpha-1-6 Mannosidase, Neuraminidase, alpha-2-3 Neuraminidase, beta 1-3 Galactosidase, and alpha-N-Acetyl-galactosaminidase

The following Examples are provided to illustrate, but not limit, the invention.

### EXAMPLES

### Example 1

### Preparation and Characterization of Small Epitope Antibodies

Five immunization polypeptides in the format of Multiple Antigenic Peptide (MAP) were designed as shown in Table 3. These sequences in combination were also used to evaluate cross-reactivity of the induced antibodies, by virtue of the inclusion in different MAPs of the same sequence in differing locations. Each of the immunization polypeptides was used to immunize 4 Balb/C mice using standard methods.

TABLE 3
Design of immunization polypeptides

| Peptide | Group | Sequence | SEQ ID NO |
|---|---|---|---|
| MAP1 | 1 | 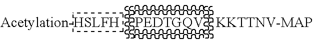 | |
| MAP2 | 2 | 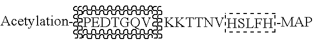 | |
| MAP3 | 3 | 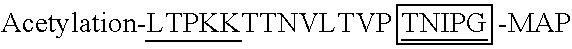 | |
| MAP4 | 4 | 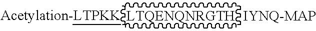 | |
| MAP5 | 5 | 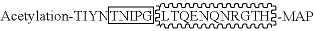 | |

Notes to Table 3:
Polypeptide MAP1: HSLFHPEDTGQV: From PSA, amino acids #79-89. KKTTNV: From Meningococcal Opa protein, containing KTT, a published 3mer antibody epitope (Malorny, Morelli et al. 1998).
Polypeptide MAP2: Alternate sequences of MAP 1.
Polypeptide MAP3: LTPKK: Motif 1 of PSA (Nagasaki, Watanabe et al. 1999). KKTTNVLTVPTNIPG: From Meningococcal Opa protein, containing two published 3mer antibody epitopes: KTT and NIP and one 4mer epitope: TNIP (Morelli, et al. (1997) Mol Microbiol 25(6): 1047-64.
Polypeptide MAP4: LTPKK: From PSA, the same as in peptide MAP3. LTQENQNRGTH: An immunogenic sequence of alpha-1-ACT selected by DNAStar computer program. IYNQ: From Meningococcal Opa protein, containing a 2mer epitope IY and four amino acids of a 5mer epitope, TIYNQ and of a 7mer epitope TPTIYNQ (Marelli, et al, id.).
Polypeptide MAP5 TIYNTNIPG: From Meningococcal Opa protein (Marelli, et al, id.). LTQENQNRGTH: The same as in peptide MAP4.

Two sets of screening polypeptides were designed: (1) 5 C-terminally biotinylated with the same sequences as the immunization polypeptides (shown in Table 4); and (2) 43 10 mer biotinylated polypeptides with sequences panning all five immunization polypeptides (shown in Table 5).

TABLE 4

Biotinylated screening polypeptides
(approximately 90% purity)

| Pep- | | |
|---|---|---|
| tide | Mers | Sequence |
| Pep1-0 | 18 | Acetylation-HSLFHPEDTGQVKKTTNV-Biotin |
| Pep2-0 | 18 | Acetylation-PEDTGQVKKTTNVHSLFH-Biotin |
| Pep3-0 | 18 | Acetylation-LTPKKTTNVLTVPTNIPG-Biotin |
| Pep4-0 | 20 | Acetylation-LTPKKLTQENQNRGTHIYNQ-Biotin |
| Pep5-0 | 20 | Acetylation-TIYNTNIPGLTQENQNRGTH-Biotin |

TABLE 5

Forty three 10mer biotinylated mapping polypeptides
(approximately 70% purity)

| | | | |
|---|---|---|---|
| | | | Position in |
| Serial | Peptide | | immunization |
| number | name | Sequence | peptides |

| | | | |
|---|---|---|---|
| 1 | Pep1-1 | Acetylated-HSLFHPEDTG-Biotin | MAP1 1-10 |
| 2 | Pep1-2 | Acetylated-SLFHPEDTGQ-Biotin | MAP1 2-11 |
| 3 | Pep1-3 | Acetylated-LFHPEDTGQV-Biotin | MAP1 3-12 |
| 4 | Pep1-4 | Acetylated-FHPEDTGQVK-Biotin | MAP1 4-13 |
| 5 | Pep1-5 | Acetylated-HPEDTGQVKK-Biotin | MAP1 5-14 |
| 6 | Pep2-1 | Acetylated-PEDTGQVKKT-Biotin | MAP1 6-15, MAP2 1-10 |
| 7 | Pep2-2 | Acetylated-EDTGQVKKTT-Biotin | MAP1 7-16, MAP2 2-11 |
| 8 | Pep2-3 | Acetylated-DTGQVKKTTN-Biotin | MAP1 8-17, MAP2 3-12 |
| 9 | Pep2-4 | Acetylated-TGQVKKTTNV-Biotin | MAP1 9-18, MAP2 4-13 |
| 10 | Pep2-5 | Acetylated-GQVKKTTNVH-Biotin | MAP2 5-14 |
| 11 | Pep2-6 | Acetylated-QVKKTTNVHS-Biotin | MAP2 6-15 |
| 12 | Pep2-7 | Acetylated-VKKTTNVHSL-Biotin | MAP2 7-16 |
| 13 | Pep2-8 | Acetylated-KKTTNVHSLF-Biotin | MAP2 8-17 |
| 14 | Pep2-9 | Acetylated-KTTNVHSLFH-Biotin | MAP2 9-18 |
| 15 | Pep3-1 | Acetylated-LTPKKTTNVL-Biotin | MAP3 1-10 |
| 16 | Pep3-2 | Acetylated-TPKKTTNVLT-Biotin | MAP3 2-11 |
| 17 | Pep3-3 | Acetylated-PKKTTNVLTV-Biotin | MAP3 3-12 |
| 18 | Pep3-4 | Acetylated-KKTTNVLTVP-Biotin | MAP3 4-13 |
| 19 | Pep3-5 | Acetylated-KTTNVLTVPT-Biotin | MAP3 5-14 |
| 20 | Pep3-6 | Acetylated-TTNVLTVPTN-Biotin | MAP3 6-15 |
| 21 | Pep3-7 | Acetylated-TNVLTVPTNI-Biotin | MAP3 7-16 |
| 22 | Pep3-8 | Acetylated-NVLTVPTNIP-Biotin | MAP3 8-17 |
| 23 | Pep3-9 | Acelylated-VLTVPTNIPG-Biotin | MAP3 9-18 |
| 24 | Pep4-1 | Acetylated-LTPKKLTQEN-Biotin | MAP4 1-10 |
| 25 | Pep4-2 | Acetylated-TPKKLTQENQ-Biotin | MAP4 2-11 |
| 26 | Pep4-3 | Acetylated-PKKLTQENQN-Biotin | MAP4 3-12 |
| 27 | Pep4-4 | Acetylated-KKLTQENQNR-Biotin | MAP4 4-13 |
| 28 | Pep4-5 | Acetylated-KLTQENQNRG-Biotin | MAP4 5-14 |
| 29 | Pep4-6 | Acetylated-LTQENQNRGT-Biotin | MAP4 6-15, MAP5 10-19 |
| 30 | Pep4-7 | Acetylated-TQENQNRGTH-Biotin | MAP4 7-16, MAP5 11-20 |
| 31 | Pep4-8 | Acetylated-QENQNRGTHI-Biotin | MAP4 8-17 |
| 32 | Pep4-9 | Acetylated-ENQNRGTHIY-Biotin | MAP4 9-18 |
| 33 | Pep4-10 | Acetylated-QENQNRGTHI-Biotin | MAP4 10-19 |
| 34 | Pep4-11 | Acetylated-ENQNRGTHIY-Biotin | MAP4 11-20 |
| 35 | Pep5-1 | Acetylated-TIYNTNIPGL-Biotin | MAP5 1-10 |
| 36 | Pep5-2 | Acetylated-IYNTNIPGLT-Biotin | MAP5 2-11 |
| 37 | Pep5-3 | Acetylated-YNTNIPGLTQ-Biotin | MAP5 3-12 |
| 38 | Pep5-4 | Acetylated-NTNIPGLTQE-Biotin | MAP5 4-13 |
| 39 | Pep5-5 | Acetylated-TNIPGLTQEN-Biotin | MAP5 5-14 |
| 40 | Pep5-6 | Acetylated-NIPGLTQENQ-Biotin | MAP5 6-15 |
| 41 | Pep5-7 | Acetylated-IPGLTQENQN-Biotin | MAP5 7-16 |
| 42 | Pep5-8 | Acetylated-PGLTQENQNR-Biotin | MAP5 8-17 |
| 43 | Pep5-9 | Acetylated-GLTQENQNRG-Biotin | MAP5 9-18 |

After a standard period of immunization, immune serum was collected from each mouse using standard methods, and tested using ELISA as follows:

ELISA plates (Corning 3369 or similar) were coated with 100 μl/well or 50 μl/well of streptavidin (Sigma Catalog No. S4762 or similar, 5 μg/ml in 50 mM carbonate buffer, pH 9.6). Plates were incubated at 4° C. overnight or at room temperature for 2 hours. Following incubation, plates were washed 3 times with PBS+0.05% Tween-20 (PBST buffer). Following washing, plates were blocked with 250 μl/well of PBST, and incubated at room temperature for 1 hour, or at 4° C. overnight. PBST was removed, and 100 μl/well or 50 μl/well of a test biotinylated polypeptide selected from Table 4, at a concentration of 5 μg/ml (diluted in PBS) was added. Plates were incubated for about 30 to 60 min at room temperature. Following incubation, plates were washed 3 times with PBST. Then, 100 μl or 50 μl/well of test serum (i.e., from test bleeds) was added, and the plates were incubated for one hour at room temperature, or overnight at 4° C. To titer immunoreactivity, the serum was generally diluted prior to testing to 1:500, 1:2000, 1:8000, or 1:32000. Following incubation, plates were washed 3 times with PBST. To detect antibody binding, a 1:10,000 dilution of goat anti-mouse IgG (and IgM)-HRP conjugate (Jackson Immuno order No. 115-036-071, or similar) was added to each well. Plates were incubated at room temperature for another hour, then washed 5 times with PBST. HRP substrate (Sigma Fast OPD,) was added and incubated in the dark at room temperature for 30-60 minutes. Plates were read at OD450 with a 96-well colorimetric detector if HRP reaction was not stopped. Alternatively, HRP reaction was stopped with 1.25M sulfuric acid, and plates were read at OD492.

12 test bleeds from Groups 1, 2, and 3 mice were tested. No immune response was observed from mice in groups 1 and 3, and these mice were not studied further. All 4 mice in group 2 showed strong immune response to screening polypeptide Pep2-0 (>1:32,000). In addition, immune sera from two of the four mice in group 2 (mice #2-1 and #2-4) showed cross-reactivity with screening polypeptides designed for groups 1 and 3 due to the sequence homology between MAP2 and MAP1/MAP3. These results were consistent with mice #2-1 and #2-4 expressing antibodies that recognize distinct and concise epitopes present within more than one screening antigen used in the ELISA assays. A test of the #2-1 and #2-4 sera versus 23 10 mer biotinylated polypeptides that span sequences of all three immunization polypeptides for group 1, 2 and 3 mice also demonstrated a broad cross-reactivity.

Eight test bleeds from groups 4-5 were tested by ELISA. Group 4 mice demonstrated a modest response to their relevant screening polypeptide, Pep4-0, while exhibiting strong cross-reactivity with Pep3-0, the screening polypeptide designed for group 3. Group 4 mice did not show substantial cross-reactivity to Pep5-0 even though there is significant sequence identity between Pep4-0 and Pep5-0. In contrast, 3 of 4 mice in group 5 (mice 5-2,5-3, 54) exhibited robust immunoreactivity to both their screening polypeptide, Pep5-0, and to the related screening polypeptide, Pep4-0. The sera from the responsive mice in group 5 did not demonstrate substantial cross-reactivity to the Pep3-0, even though there is a 5 amino acid block of sequence identity. A test of the #5-2 and 5-3 sera versus 23 10 mer biotinylated polypeptides that span sequences of all three immunization polypeptides for group 4 and 5 mice demonstrated two broad but distinctive reaction patterns with the mapping polypeptides spanning sequences of immunization polypeptides for groups 4 and 5 mice.

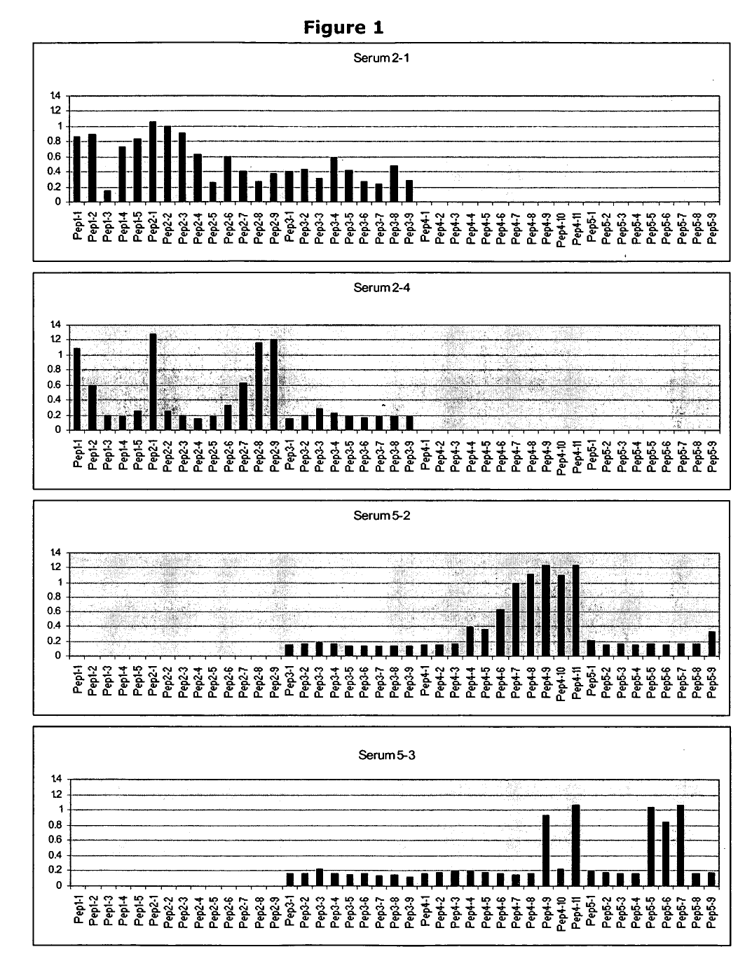

Group 2, mice #1 and #4, and Group 5, mice #2 and #3, showed the best immune responses, as summarized in Table 6 and FIG. 1. These mice were selected for hybridoma fusions.

TABLE 6
Immunoreactivity and cross-reactivity of
selected mice in Groups 2 and 5 to
screening polypeptides 1-5.

| Mouse | Peptide 1 | Peptide 2 | Peptide 3 | Peptide 4 | Peptide 5 |
|---|---|---|---|---|---|
| 2-1 | 0.726 | 0.850 | 0.323 | Not tested | Not tested |
| 2-2 | 0.250 | 1.167 | 0.213 | Not tested | Not tested |
| 2-3 | 0.222 | 0.685 | 0.141 | Not tested | Not tested |
| 2-4 | 0.776 | 0.970 | 0.353 | Not tested | Not tested |
| 5-1 | Not tested | Not tested | 0.178 | 0.28 | 0.979 |
| 5-2 | Not tested | Not tested | 0.146 | 1.714 | 1.548 |
| 5-3 | Not tested | Not tested | 0.13 | 1.479 | 1.773 |
| 5-4 | Not tested | Not tested | 0.128 | 1.915 | 1.464 |

The animals were sacrificed, the lymph nodes and spleens harvested, then B cell hybridoma fusions using P3 mouse myeloma cell line as a fusion partner were generated using standard methods. Fusions were plated and incubated for 11-14 days before screening.

In the first round of screening, hybridomas from group 2 and 5 mice were analyzed by ELISA in 96 well plates, essentially as described above, using the corresponding screening polypeptides, 2-0 and 5-0. Following several rounds of screening, 48 positive hybridoma lines were identified and transferred to 24 well plates for expansion and additional characterization including epitope mapping. Of the 48 positive lines, 33 were derived from the Group 2 animals that received the MAP2 immunogen while the remaining 15 originated from the Group 5 animals. Most of the hybridoma lines (˜94%) were the fusion products of B cells harvested from the spleen. Thirteen of the 48 hybridoma lines expressed IgG, 25 expressed IgM, and the remaining 10 hybridoma lines were expressing both IgG and IgM or were not expressing either IgG or IgM and were therefore expressing either IgA or IgE.

In the second round of screening, hybridomas selected for expansion were re-tested against the relevant screening polypeptide (either polypeptide 2-0 or polypeptide 5-0). 13 of the 48 hybridomas characterized after the 24 well expansion phase exhibited sequence specific binding to the screening polypeptide 2-0. Other hybridomas bound non-specifically (i.e., bound a variety of oligopeptide sequences), failed to bind (reflecting either a false positive or clonal instability and loss during the transfer and subsequent propagation in 24 well plates) or bound control wells containing BSA.

The 13 hybridomas that specifically bound to screening polypeptide 2-0 were epitope mapped using ELISA as described above, using 3 different sets of 10mer C-terminal biotinylated mapping polypeptides: polypeptides 1-1 to 1-5; 2-1 to 2-9; and 3-1 to 3-9 (see Table 5). 10 of the 12 hybridoma lines exhibited maximum reactivity with a single mapping polypeptide, 2-1, and that hybridomas 2.03 and 2.11 showed strong binding to different overlapping sets of mapping polypeptides, polypeptides 2-1 through 2-3 and 2-7 through 2-9. Because these data showed strong reactivity to a single mapping polypeptide for most hybridoma lines, we considered the possibility that steric hindrance associated with immobilization of the mapping polypeptides (specifically, biotin-avidin immobilization) was preventing antibody binding to the epitope present within a cognate series of 10mers, thus potentially biasing the ELISA epitope map results. Thus, we evaluated epitope specificity using a competitive binding assay.

Individual mapping polypeptides were evaluated for their ability to inhibit antibody binding to the 2-0 screening polypeptide affixed to streptavidin-coated 96 well plates. In this format, the 10mer mapping polypeptides were not tethered within the binding pocket of streptavidin and consequently should not be sterically hindered from interacting with a reactive antibody present within the set of 13 hybridomas. Competition experiments were performed using standard methods using the 2-0 screening polypeptide affixed to streptavidin-coated 96 well plates and 10mer mapping polypeptide added to each well.

Using the competitive binding assay, the epitopes recognized by 10 of the 13 hybridomas were determined. Eight of the hybridomas were specific for the epitope PEDTG, hybridoma 2.03 was specific for epitope DTG and hybridoma 2.11 recognized the epitope KKTTN. Hybridoma 2.31 exhibited a complex inhibition pattern suggesting that this line is a mixture of 2 or more specificities and should be subcloned to segregate the individual reactivities. Finally, hybridomas 1.02 and 2.12 showed poor discrimination in the competitive inhibition assay. The results of this analysis are summarized in Table 7.

TABLE 7
Epitopes Predicted by Competitive Inhibition

Pattern of 1.01, 2.01, 2.04, 2.06, 2.07,
2.08, 2.10 and 2.23: PEDTG

| | |
|---|---|
| P1-1 | HSLFHPEDTG |
| P1-2 | SLFHPEDTGQ |
| P1-5 | HPEDTGQVKK |
| P2-1 | PEDTGQVKKT |

2.03 Pattern: DTG

| | |
|---|---|
| P1-1 | HSLFHPEDTG |
| P1-2 | SLFHPEDTGQ |
| P1-3 | LFHPEDTGQV |
| P1-4 | FHPEDTGQVK |
| P1-5 | HPEDTGQVKK |
| P2-1 | PEDTGQVKKT |
| P2-2 | EDTGQVKKTT |
| P2-3 | DTGQVKKTTN |

2.11 Pattern: KKTTN

| | |
|---|---|
| P1-4 | FHPEDTGQVK ??? |
| P2-3 | DTGQVKKTTN |
| P2-4 | TGQVKKTTNV |
| P2-5 | GQVKKTTNVH |
| P2-6 | QVKKTTNVHS |
| P2-7 | VKKTTNVHSL |
| P2-8 | |
| P2-9 | |
| P3-1 | LTPKKTTNVL |
| P3-2 | TPKKTTNVLT |
| P3-3 | PKKTTNVLTV |
| P3-4 | KKTTNVLTVP |

2.31 Pattern: A mixture of two clones?

| | |
|---|---|
| P1-1 | HSLFHPEDTG |
| P1-2 | SLFHPEDTGQ |
| P1-5 | HPEDTGQVKK |
| P2-1 | PEDTGQVKKT |
| P2-7 | VKKTTNVHSL |
| P2-8 | KKTTNVHSLF |
| P2-9 | KTTNVHSLFH |
| P3-2 | TPKKTTNVLT |
| P3-3 | PKKTTNVLTV |

1.02 and 2.12 Pattern: Pattern is unclear

The competitive binding assays were repeated twice, and it was confirmed that hybridoma 2.11 recognized the epitope KTTN, not the epitope KKTTN as suggested in the preliminary experiments. The epitope competitive binding assays confirmed the epitope characterization described above for the other hybridomas. The results of this updated analysis are summarized in Table 8. Hybridomas 2.03 (also called DA001-2.03), 2.04 (DA001-2.04) and so called DA001-2.11) are being prepared for deposit at the ATCC.

TABLE 8

Updated and Confirmed Table of Epitopes Predicted
by Competitive Inhibition

Pattern of 1.01, 2.01, 2.04, 2.06, 2.07,
2.08, 2.10 and 2.23: PEDTG

| | |
|---|---|
| P1-1 | HSLFHPEDTG |
| P1-2 | SLFHPEDTGQ |
| P1-5 | HPEDTGQVKK |
| P2-1 | PEDTGQVKKT |

2.03 Pattern: DTG

| | |
|---|---|
| P1-1 | HSLFHPEDTG |
| P1-2 | SLFHPEDTGQ |
| P1-3 | LFHPEDTGQV |
| P1-4 | FHPEDTGQVK |
| P1-5 | HPEDTGQVKK |
| P2-1 | PEDTGQVKKT |
| P2-2 | EDTGQVKKTT |
| P2-3 | DTGQVKKTTN |

2.11 Pattern: KKTTN

| | |
|---|---|
| P1-4 | FHPEDTGQVK ??? |
| P2-3 | DTGQVKKTTN |
| P2-4 | TGQVKKTTNV |
| P2-5 | GQVKKTTNVH |
| P2-6 | QVKKTTNVHS |
| P2-7 | VKKTTNVHSL |
| P2-8 | |
| P2-9 | |
| P3-1 | LTPKKTTNVL |
| P3-2 | TPKKTTNVLT |
| P3-3 | PKKTTNVLTV |
| P3-4 | KKTTNVLTVP |

2.31 Pattern: A mixture of two clones?

| | |
|---|---|
| P1-1 | HSLFHPEDTG |
| P1-2 | SLFHPEDTGQ |
| P1-5 | HPEDTGQVKK |
| P2-1 | PEDTGQVKKT |
| P2-7 | VKKTTNVHSL |
| P2-8 | KKTTNVHSLF |
| P2-9 | KTTNVHSLFH |
| P3-2 | TPKKTTNVLT |
| P3-3 | PKKTTNVLTV |

1.02 and 2.12 Pattern: Pattern is unclear

### Example 2

### Preparation of Small Epitope Antibodies

An approach to identify antibodies based on phage display antibody screening was performed. Five peptide sequences used for the selection of positive antibodies are shown in Table 9. These sequences in combination were also used to evaluate cross-reactivity of the selected antibodies.

TABLE 9
Design of screening polypeptides

| | |
|---|---|
| Peptide | Sequence |

| | |
|---|---|
| P1 | CXXXXXDTGXXXXXX |
| P6 | CXXXXXDTGXXXXXX |
| P7 | CXXXXXAQVXXXXXX |
| P8 | CXXXXXIARXXXXXX |
| P9 | CXXXXXLSHXXXXXX |

Note to Table 9: The letter ‘X’ denotes a mixture of the naturally-occurring L-amino acids excluding cysteine, methionine, and tryptophan.

Positives were selected after six rounds of enrichment. The results of phage ELISA screens against the five screening peptides is shown in Table 10. A total of 96 phage were screened for P1; 48 were screened for polypeptides P6-P9. In all cases, positive phage were identified above background.

TABLE 10
Reactivity of enriched phage against screening polypeptides

| Polypeptide 1 | | Polypeptide 6 | | Polypeptide 7 | | Polypeptide 8 | | Polypeptide 9 | |
|---|---|---|---|---|---|---|---|---|---|
| Phage | OD | Phage | OD | Phage | OD | Phage | OD | Phage | OD |
| L50P1_1 | 0.0781 | L50P6_1 | 1.6477 | I50P7_1 | 0.0791 | L50P8_1 | 0.5249 | L50P9_1 | 0.0813 |
| L50P1_2 | 0.0737 | L50P6_2 | 1.6612 | I50P7_2 | 0.3119 | L50P8_2 | 0.4247 | L50P9_2 | 0.4743 |
| L50P1_3 | 0.0684 | L50P6_3 | 1.5365 | I50P7_3 | 0.2111 | L50P8_3 | 0.8174 | L50P9_3 | 0.6882 |
| L50P1_4 | 0.3906 | L50P6_4 | 1.4133 | I50P7_4 | 1.6251 | L50P8_4 | 0.6231 | L50P9_4 | 0.5747 |
| L50P1_5 | 0.3333 | L50P6_5 | 0.9797 | I50P7_5 | 1.3357 | L50P8_5 | 0.5497 | L50P9_5 | 0.4527 |
| L50P1_6 | 0.0667 | L50P6_6 | 0.1036 | I50P7_6 | 0.2128 | L50P8_6 | 0.7834 | L50P9_6 | 0.6045 |
| L50P1_7 | 0.0668 | L50P6_7 | 0.5592 | I50P7_7 | 1.4445 | L50P8_7 | 0.4143 | L50P9_7 | 0.0944 |
| L50P1_8 | 0.0689 | L50P6_8 | 1.5017 | I50P7_8 | 0.0694 | L50P8_8 | 0.8192 | L50P9_8 | 0.0762 |
| L50P1_9 | 0.0714 | L50P6_9 | 1.1022 | I50P7_9 | 0.7113 | L50P8_9 | 0.5725 | L50P9_9 | 0.3449 |
| L50P1_10 | 0.0683 | L50P6_10 | 1.1577 | I50P7_10 | 0.1787 | L50P8_10 | 0.6108 | L50P9_10 | 0.0721 |
| L50P1_11 | 0.0813 | L50P6_11 | 0.4477 | I50P7_11 | 0.1912 | L50P8_11 | 0.2095 | L50P9_11 | 0.6566 |
| L50P1_12 | 0.1168 | L50P6_12 | 1.2041 | I50P7_12 | 0.1158 | L50P8_12 | 0.6757 | L50P9_12 | 0.0831 |
| L50P1_13 | 0.0717 | L50P6_13 | 1.6751 | I50P7_13 | 0.0729 | L50P8_13 | 0.5143 | L50P9_13 | 0.4898 |
| L50P1_14 | 0.4481 | L50P6_14 | 1.1052 | I50P7_14 | 0.1238 | L50P8_14 | 0.659 | L50P9_14 | 0.5458 |
| L50P1_15 | 0.6361 | L50P6_15 | 0.218 | I50P7_15 | 0.0679 | L50P8_15 | 1.0582 | L50P9_15 | 0.0702 |
| L50P1_16 | 0.2818 | L50P6_16 | 0.0787 | I50P7_16 | 0.0688 | L50P8_16 | 0.8478 | L50P9_16 | 0.4297 |
| L50P1_17 | 0.4623 | L50P6_17 | 0.066 | I50P7_17 | 0.0847 | L50P8_17 | 0.7276 | L50P9_17 | 0.3535 |
| L50P1_18 | 0.0614 | L50P6_18 | 0.1961 | I50P7_18 | 1.0256 | L50P8_18 | 0.7266 | L50P9_18 | 0.0757 |
| L50P1_19 | 0.0595 | L50P6_19 | 1.1042 | I50P7_19 | 1.5344 | L50P8_19 | 0.6607 | L50P9_19 | 0.07 |
| L50P1_20 | 0.0821 | L50P6_20 | 0.0618 | I50P7_20 | 0.4507 | L50P8_20 | 0.8016 | L50P9_20 | 0.547 |
| L50P1_21 | 0.08 | L50P6_21 | 1.155 | I50P7_21 | 0.2637 | L50P8_21 | 0.754 | L50P9_21 | 0.5593 |
| L50P1_22 | 0.0632 | L50P6_22 | 1.4566 | I50P7_22 | 0.1088 | L50P8_22 | 0.4702 | L50P9_22 | 0.6068 |
| L50P1_23 | 0.0643 | L50P6_23 | 0.129 | I50P7_23 | 1.0236 | L50P8_23 | 0.3573 | L50P9_23 | 0.5225 |
| L50P1_24 | 0.0817 | L50P6_24 | 1.2605 | I50P7_24 | 0.1236 | L50P8_24 | 0.7595 | L50P9_24 | 0.8072 |
| L50P1_25 | 0.0917 | L50P6_25 | 0.0583 | I50P7_25 | 0.0965 | L50P8_25 | 0.7424 | L50P9_25 | 0.5658 |
| L50P1_26 | 0.0791 | L50P6_26 | 0.0848 | I50P7_26 | 0.898 | L50P8_26 | 0.7334 | L50P9_26 | 0.0758 |
| L50P1_27 | 0.0619 | L50P6_27 | 0.0805 | I50P7_27 | 0.1256 | L50P8_27 | 0.7748 | L50P9_27 | 0.3991 |
| L50P1_28 | 0.4974 | L50P6_28 | 1.5586 | I50P7_28 | 0.7453 | L50P8_28 | 0.6577 | L50P9_28 | 0.5235 |
| L50P1_29 | 0.0596 | L50P6_29 | 0.0778 | I50P7_29 | 0.1149 | L50P8_29 | 0.5632 | L50P9_29 | 0.0699 |
| L50P1_30 | 0.0582 | L50P6_30 | 1.5647 | I50P7_30 | 0.076 | L50P8_30 | 0.5071 | L50P9_30 | 0.516 |
| L50P1_31 | 0.4591 | L50P6_31 | 0.0962 | I50P7_31 | 1.4382 | L50P8_31 | 0.5892 | L50P9_31 | 0.2835 |
| L50P1_32 | 0.0566 | L50P6_32 | 0.0603 | I50P7_32 | 1.5916 | L50P8_32 | 0.6455 | L50P9_32 | 0.0733 |
| L50P1_33 | 0.0622 | L50P6_33 | 0.0815 | I50P7_33 | 0.8539 | L50P8_33 | 0.4008 | L50P9_33 | 0.5253 |
| L50P1_34 | 0.0584 | L50P6_34 | 0.1512 | I50P7_34 | 1.0193 | L50P8_34 | 0.4515 | L50P9_34 | 0.5407 |
| L50P1_35 | 0.7212 | L50P6_35 | 0.1344 | I50P7_35 | 0.1178 | L50P8_35 | 0.4302 | L50P9_35 | 0.0744 |
| L50P1_36 | 0.0843 | L50P6_36 | 0.1644 | I50P7_36 | 1.2705 | L50P8_36 | 0.3179 | L50P9_36 | 0.613 |
| L50P1_37 | 0.4181 | L50P6_37 | 1.2164 | I50P7_37 | 0.4899 | L50P8_37 | 0.4526 | L50P9_37 | 0.5239 |
| L50P1_38 | 0.4914 | L50P6_38 | 1.3835 | I50P7_38 | 0.142 | L50P8_38 | 0.7307 | L50P9_38 | 0.1844 |
| L50P1_39 | 0.0607 | L50P6_39 | 0.1062 | I50P7_39 | 0.5033 | L50P8_39 | 0.7737 | L50P9_39 | 0.0804 |
| L50P1_40 | 0.5813 | L50P6_40 | 0.0615 | I50P7_40 | 0.7136 | L50P8_40 | 0.6617 | L50P9_40 | 0.3825 |
| L50P1_41 | 0.3373 | L50P6_41 | 1.3978 | I50P7_41 | 0.2031 | L50P8_41 | 0.6766 | L50P9_41 | 0.0748 |
| L50P1_42 | 0.0561 | L50P6_42 | 0.0758 | I50P7_42 | 0.0669 | L50P8_42 | 0.6741 | L50P9_42 | 0.2942 |
| L50P1_43 | 0.3979 | L50P6_43 | 0.0831 | I50P7_43 | 0.1266 | L50P8_43 | 0.6942 | L50P9_43 | 0.0707 |
| L50P1_44 | 0.0587 | L50P6_44 | 1.5906 | I50P7_44 | 0.0693 | L50P8_44 | 0.6275 | L50P9_44 | 0.0722 |
| L50P1_45 | 0.0576 | L50P6_45 | 0.081 | I50P7_45 | 0.1209 | L50P8_45 | 0.3312 | L50P9_45 | 0.5045 |
| L50P1_46 | 0.0699 | L50P6_46 | 1.4628 | I50P7_46 | 0.4689 | L50P8_46 | 0.3838 | L50P9_46 | 0.2859 |
| L50P1_47 | 0.4785 | L50P6_47 | 0.1462 | I50P7_47 | 0.0686 | L50P8_47 | 0.3922 | L50P9_47 | 0.4253 |
| L50P1_48 | 0.6597 | L50P6_48 | 0.0738 | Neg Control | 0.0634 | L50P8_48 | 0.5962 | Neg Control | 0.1297 |
| Neg Control | 0.0738 | Neg Control | 0.1297 | | | Neg Control | 0.1297 | | |

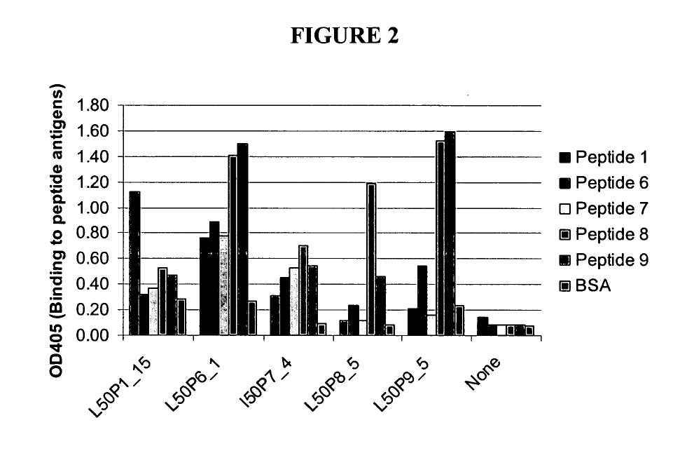

In a secondary screen of positives identified in the primary screen, a phage ELISA assay was done against all five polypeptides. Up to five positives were selected for the secondary screen. FIG. 2 shows the results of the most selective clones using this assay. All five positives yielded significant signal to polypeptide above BSA, and the phage selected from P1 (L50P1_{—}15), P8 (L50P8_{—}5), and P9 (L50P9_{—}5) appear to show specificity in this semi-quantitative assay.

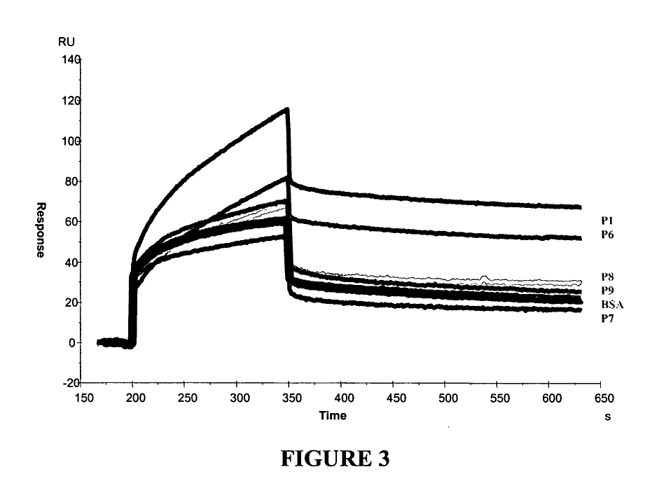

The reactive antibody for L50P1_{—}15 was subcloned into a vector for bacterial expression of single chain antibodies. The crude periplasmic preparation was analyzed using a surface plasmon resonance (SPR) biosensor assay to monitor the formation of complex association and the dissociation of the protein from immobilized peptides (Malmborg et al, 1995). FIG. 3 shows the SPR profile of single chain antibody against the five polypeptides and BSA. The antibody has the highest affinity for peptide 1, with an estimated K_{d} of 2×10⁻⁸.

### Example 3

### Protein Profiling and Biomarker Development

In one exemplary method for protein profiling, serums derived from healthy and affected individuals for a particular disease of clinical interest are subjected to: (a) debulking of the most abundant protein constituents; (b) deglycosylation of the less abundant proteins that remain; (c) reduction and alkylation of cysteine residues present in the debulked proteome; (d) digestion of the debulked proteome to completion; (e) fractionation of the resulting peptide fragments with small epitope antibodies as described above; and (f) comparison of the composition and relative abundance of peptide constituents from epitope enriched fractions derived from healthy and affected patients to identify candidate biomarkers associated with a specific disease.

Fractionation with small epitope antibodies is performed in parallel with a set of approximately 100 small epitope antibodies of different specificities. Each antibody is chosen based on a set of criteria including epitope size, epitope abundance in the serum proteome, specificity, affinity, and sampling redundancy. The epitopes recognized by the antibodies are predominantly 3mers, although some are 4mers or 5mers that satisfy the abundance criteria, with each epitope occurring in 0.5-3% of the constituents of the serum proteome. Each antibody recognizes its cognate epitope in a context-independent manner and with high affinity. The complete set of small epitope antibodies used for fractionation provides 3-5 fold sampling redundancy to accommodate the variability expected in both expression levels for different proteins and capture efficiencies for each antibody in the set.

Mass spectroscopy is used to analyze the peptide composition and peptide constituent expression levels for each small epitope antibody fraction. Biomarkers are identified that are differentially expressed in healthy and diseased individuals. ELISA assays are developed that can discriminate between healthy and affected individuals based on specific levels of identified biomarkers present in plasma or serum.

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention, which is delineated by the appended claims.

All publications, patents, and patent applications cited herein are hereby incorporated by reference in their entireties for all purposes and to the same extent as if each individual publication, patent, or patent application were specifically and individually indicated to be so incorporated by reference.

1. A method for reducing the complexity of a sample that comprises a mixture of proteins, said method comprising separating a small epitope antibody-protein complex, wherein proteins comprising an epitope bound by the small epitope antibody are enriched.

2. A method according to claim 1, further comprising separating protein from the antibody-protein complex.

3. A method according to claim 2, further comprising contacting the protein separated from the antibody-protein complex with a protein cleaving agent to form polypeptide fragments.

4. A method according to claim 3, wherein the protein cleaving agent comprises a protease.

5. A method according to claim 3, wherein the protein cleaving agent comprises a chemical agent.

6. A method according to claim 1, further comprising contacting the antibody-protein complex with a protein cleaving agent to form polypeptide fragments.

7. A method according to claim 6, wherein the protein cleaving agent comprises a protease.

8. A method according to claim 6, wherein the protein cleaving agent comprises a chemical agent.

9. A method according to claim 1, further comprising contacting the sample with a protein cleaving agent to form polypeptide fragments prior to formation of said small epitope antibody-protein complex.

10. A method according to claim 9, further comprising separating polypeptide fragments from the antibody-protein complex.

11. A method according to claim 10, wherein the protein cleaving agent comprises a protease.

12. A method according to claim 11, wherein the protein cleaving agent comprises a chemical agent.

13. An antibody-protein complex prepared according to the method of claim 1.

14. A protein prepared according to the method of claim 2.

15. A polypeptide fragment prepared according to the method of claim 3.

16. A polypeptide fragment prepared according to the method of claim 6.

17. A polypeptide fragment prepared according to the method of claim 10.

18. A method according to claim 1, wherein the small epitope antibody binds an epitope consisting of about 3 to about 5 amino acids.

19. A method according to claim 1, wherein the sample is contacted with a plurality of small epitope antibodies to form a plurality of antibody-protein complexes.

20. A method according to claim 19, wherein the small epitope antibodies are immobilized on a solid matrix.

21. A method according to claim 19, wherein the small epitope antibodies are detectably labeled.

22. A method according to claim 19, wherein said contacting of the sample with the small epitope antibodies is performed in parallel.

23. A method according to claim 19, wherein said contacting of the sample with the small epitope antibodies is performed serially.

24. A method according to claim 1, wherein the sample is contacted with at least about 100 small epitope antibodies.

25. A method for reducing the complexity of a sample that comprises a mixture of proteins, said method comprising:
(a) contacting the sample with at least one small epitope antibody to form an antibody-protein complex; and
(b) separating said antibody-protein complex from unbound protein in the sample.

26. A method according to claim 25, wherein steps (a) and (b) are performed sequentially.

27. A method according to claim 25, wherein steps (a) and (b) are performed simultaneously.

28. A method according to claim 25, wherein the small epitope antibody binds an epitope consisting of about 3 to about 5 amino acids.

29. A method according to claim 25, wherein the at least one small epitope antibody comprises at least about 100 small epitope antibodies.

30. A method according to claim 25, further comprising separating protein from the antibody-protein complex.

31. A method for determining presence or absence of a protein of interest in a sample, said method comprising detecting the protein of interest, if any, in an enriched protein fraction, wherein the enriched protein fraction is prepared by the method of claim 1, and wherein detection of the protein of interest indicates presence of the protein in the sample.

32. A method according to claim 31, wherein said detection comprises mass spectrometry.

33. A method for determining the amount of a protein of interest in a sample, said method comprising quantifying the amount of the protein of interest in an enriched protein fraction, wherein the enriched protein fraction is prepared by the method of claim 1.

34. A method according to claim 33, wherein said quantifying comprises mass spectrometry.

35. A method for identifying a protein in a small epitope antibody-protein complex, wherein the small epitope antibody-protein complex is prepared according to claim 1.

36. A method according to claim 35, wherein said identifying comprises mass spectrometry.

37. A method for identification of a biomarker, said method comprising comparing the proteins in the two or more enriched protein fractions, wherein each of the enriched protein fractions is prepared from a sample according to the method of claim 1.

38. A method according to claim 37, wherein the two or more samples comprise samples from at least one individual who has a disease condition and at least one individual who does not have the disease condition, and wherein presence or absence of the biomarker is indicative of the disease condition.

39. A method for determining presence or absence of a disease condition in an individual, the method comprising determining the level of a biomarker in a sample from the individual, wherein the biomarker is identified according to the method of claim 38, and wherein the level of the biomarker is indicative of the presence or absence of the disease condition.

40. A method according to claim 37, wherein the two or more samples comprise samples from at least one individual who has received treatment for a disease condition and at least one individual who has not received treatment for the disease condition, and wherein presence or absence of the biomarker is indicative of efficacy of the treatment.

41. A method for determining efficacy of treatment for a disease condition in an individual, the method comprising determining the level of a biomarker in a sample from the individual, wherein the biomarker is identified according to the method of claim 40, and wherein the level of the biomarker is indicative of the efficacy of treatment.

42. A method according to claim 37, wherein the two or more samples comprise samples from at least one individual who has been exposed to a toxin or pathogen and at least one individual who has not been exposed to the toxin or pathogen, and wherein presence or absence of the biomarker is indicative of exposure of an individual to the toxin or pathogen.

43. A method for determining exposure of an individual to a toxin or pathogen, the method comprising determining the level of a biomarker in a sample from the individual, wherein the biomarker is identified according to the method of claim 42, and wherein the level of the biomarker is indicative of exposure to the toxin or pathogen.

44. A composition comprising a plurality of small epitope antibodies.

45. A composition according to claim 44, wherein said plurality of small epitope antibodies binds epitopes consisting of about 3 to about 5 amino acids.

46. A composition according to claim 45, wherein said small epitope antibodies are detectably labeled.

47. A composition according to claim 44, wherein said plurality of small epitope antibodies comprises at least about 100 small epitope antibodies.

48. A kit comprising a plurality of small epitope antibodies.

49. A kit according to claim 48, wherein said plurality of small epitope antibodies binds epitopes consisting of about 3 to about 5 amino acids.

50. A kit according to claim 48, wherein said small epitope antibodies are detectably labeled.

51. A kit according to claim 48, wherein said plurality of small epitope antibodies comprises at least about 100 small epitope antibodies.


SEQUENCE LISTING (74 sequences)

SEQ ID NO: 1 - protein, 4 aa, Neisseria meningitidis opa proteins
NRQD

SEQ ID NO: 2 - protein, 4 aa, Neisseria meningitidis opa proteins
TTFL

SEQ ID NO: 3 - protein, 4 aa, Porcine ZP3 beta
WQDE

SEQ ID NO: 4 - protein, 4 aa, Gp120 of HIV-1
GPGR

SEQ ID NO: 5 - protein, 4 aa, Crotoxin
  VARIANT 1: Xaa = Asp or Ser
XGYX

SEQ ID NO: 6 - protein, 18 aa, Artificial Sequence
  VARIANT 1: N-terminal residue is acetylated.
HSLFHPEDTGQVKKTTNV

SEQ ID NO: 7 - protein, 18 aa, Artificial Sequence
  VARIANT 1: N-terminal residue is acetylated.
PEDTGQVKKTTNVHSLFH

SEQ ID NO: 8 - protein, 18 aa, Artificial Sequence
  VARIANT 1: N-terminal residue is acetylated.
LTPKKTTNVLTVPTNIPG

SEQ ID NO: 9 - protein, 20 aa, Artificial Sequence
  VARIANT 1: N-terminal residue is acetylated.
LTPKKLTQENQNRGTHIYNQ

SEQ ID NO: 10 - protein, 20 aa, Artificial Sequence
  VARIANT 1: N-terminal residue is acetylated.
TIYNTNIPGLTQENQNRGTH

SEQ ID NO: 11 - protein, 12 aa, Homo sapiens
HSLFHPEDTGQV

SEQ ID NO: 12 - protein, 6 aa, Neisseria meningitidis opa proteins
KKTTNV

SEQ ID NO: 13 - protein, 5 aa, Homo sapiens PSA
LTPKK

SEQ ID NO: 14 - protein, 15 aa, Neisseria meningitidis opa proteins
KKTTNVLTVPTNIPG

SEQ ID NO: 15 - protein, 4 aa, Neisseria meningitidis opa proteins
TNIP

SEQ ID NO: 16 - protein, 11 aa, Homo sapiens
LTQENQNRGTH

SEQ ID NO: 17 - protein, 4 aa, Neisseria meningitidis opa proteins
IYNQ

SEQ ID NO: 18 - protein, 5 aa, Neisseria meningitidis opa proteins
TIYNQ

SEQ ID NO: 19 - protein, 7 aa, Neisseria meningitidis opa proteins
TPTIYNQ

SEQ ID NO: 20 - protein, 9 aa, Neisseria meningitidis opa proteins
TIYNTNIPG

SEQ ID NO: 21 - protein, 18 aa, Neisseria meningitidis opa proteins
  VARIANT 1: N-terminal residue is acetylated.
HSLFHPEDTGQVKKTTNV

SEQ ID NO: 22 - protein, 18 aa, Neisseria meningitidis opa proteins
  VARIANT 1: N-terminal residue is acetylated.
PEDTGQVKKTTNVHSLFH

SEQ ID NO: 23 - protein, 18 aa, Neisseria meningitidis opa proteins
  VARIANT 1: N-terminal residue is acetylated.
LTPKKTTNVLTVPTNIPG

SEQ ID NO: 24 - protein, 20 aa, Neisseria meningitidis opa proteins
  VARIANT 1: N-terminal residue is acetylated.
LTPKKLTQENQNRGTHIYNQ

SEQ ID NO: 25 - protein, 20 aa, Neisseria meningitidis opa proteins
  VARIANT 1: N-terminal residue is acetylated.
TIYNTNIPGLTQENQNRGTH

SEQ ID NO: 26 - protein, 11 aa, Homo sapiens PSA
  VARIANT 1: N-terminal residue is acetylated.
HSLFHFPEDTG

SEQ ID NO: 27 - protein, 10 aa, Homo sapiens PSA
  VARIANT 1: N-terminal residue is acetylated.
SLFHPEDTGQ

SEQ ID NO: 28 - protein, 10 aa, Homo sapiens PSA
  VARIANT 1: N-terminal residue is acetylated.
LFHPEDTGQV

SEQ ID NO: 29 - protein, 10 aa, Artificial Sequence
  Combination of Homo sapiens PSA and Neisseria meningitidis opa proteins.
FHPEDTGQVK

SEQ ID NO: 30 - protein, 10 aa, Artificial Sequence
  Combination of Homo sapiens PSA and Neisseria meningitidis opa proteins.
HPEDTGQVKK

SEQ ID NO: 31 - protein, 10 aa, Artificial Sequence
  Combination of Homo sapiens PSA and Neisseria meningitidis opa proteins.
PEDTGQVKKT

SEQ ID NO: 32 - protein, 10 aa, Artificial Sequence
  Combination of Homo sapiens PSA and Neisseria meningitidis opa proteins.
EDTGQVKKTT

SEQ ID NO: 33 - protein, 10 aa, Artificial Sequence
  Combination of Homo sapiens PSA and Neisseria meningitidis opa proteins.
DTGQVKKTTN

SEQ ID NO: 34 - protein, 10 aa, Artificial Sequence
  Combination of Homo sapiens PSA and Neisseria meningitidis opa proteins.
TGQVKKTTNV

SEQ ID NO: 35 - protein, 10 aa, Artificial Sequence
  Combination of Homo sapiens PSA and Neisseria meningitidis opa proteins.
GQVKKTTNVH

SEQ ID NO: 36 - protein, 10 aa, Artificial Sequence
  Combination of Homo sapiens PSA and Neisseria meningitidis opa proteins.
QVKKTTNVHS

SEQ ID NO: 37 - protein, 10 aa, Artificial Sequence
  Combination of Homo sapiens PSA and Neisseria meningitidis opa proteins.
VKKTTNVHSL

SEQ ID NO: 38 - protein, 10 aa, Artificial Sequence
  Combination of Homo sapiens PSA and Neisseria meningitidis opa proteins.
KKTTNVHSLF

SEQ ID NO: 39 - protein, 10 aa, Artificial Sequence
  Combination of Homo sapiens PSA and Neisseria meningitidis opa proteins.
KTTNVHSLFH

SEQ ID NO: 40 - protein, 10 aa, Artificial Sequence
  Combination of Homo sapiens PSA and Neisseria meningitidis opa proteins.
LTPKKTTNVL

SEQ ID NO: 41 - protein, 10 aa, Artificial Sequence
  Combination of Homo sapiens PSA and Neisseria meningitidis opa proteins.
TPKKTTNVLT

SEQ ID NO: 42 - protein, 10 aa, Artificial Sequence
  Combination of Homo sapiens PSA and Neisseria meningitidis opa proteins.
PKKTTNVLTV

SEQ ID NO: 43 - protein, 10 aa, Neisseria meningitidis opa protein
  VARIANT 1: N-terminal residue is acetylated.
KKTTNVLTVP

SEQ ID NO: 44 - protein, 10 aa, Neisseria meningitidis opa protein
  VARIANT 1: N-terminal residue is acetylated.
KTTNVLTVPT

SEQ ID NO: 45 - protein, 10 aa, Neisseria meningitidis opa protein
  VARIANT 1: N-terminal residue is acetylated.
TTNVLTVPTN

SEQ ID NO: 46 - protein, 10 aa, Neisseria meningitidis opa protein
  VARIANT 1: N-terminal residue is acetylated.
TNVLTVPTNI

SEQ ID NO: 47 - protein, 10 aa, Neisseria meningitidis opa protein
  VARIANT 1: N-terminal residue is acetylated.
NVLTVPTNIP

SEQ ID NO: 48 - protein, 10 aa, Neisseria meningitidis opa proteins
  VARIANT 1: N-terminal residue is acetylated.
VLTVPTNIPG

SEQ ID NO: 49 - protein, 10 aa, Homo sapiens PSA and ACT
  VARIANT 1: N-terminal residue is acetylated.
LTPKKLTQEN

SEQ ID NO: 50 - protein, 10 aa, Homo sapiens PSA and ACT
  VARIANT 1: N-terminal residue is acetylated.
TPKKLTQENQ

SEQ ID NO: 51 - protein, 10 aa, Homo sapiens PSA and ACT
  VARIANT 1: N-terminal residue is acetylated.
PKKLTQENQN

SEQ ID NO: 52 - protein, 10 aa, Homo sapiens PSA and ACT
  VARIANT 1: N-terminal residue is acetylated.
KKLTQENQNR

SEQ ID NO: 53 - protein, 10 aa, Homo sapiens PSA and ACT
  VARIANT 1: N-terminal residue is acetylated.
KLTQENQNRG

SEQ ID NO: 54 - protein, 10 aa, Homo sapiens ACT
  VARIANT 1: N-terminal residue is acetylated.
LTQENQNRGT

SEQ ID NO: 55 - protein, 10 aa, Homo sapiens ACT
  VARIANT 1: N-terminal residue is acetylated.
TQENQNRGTH

SEQ ID NO: 56 - protein, 10 aa, Artificial Sequence
  Combination of Homo sapiens ACT and Neisseria meningitidis opa proteins
QENQNRGTHI

SEQ ID NO: 57 - protein, 10 aa, Artificial Sequence
  Combination of Homo sapiens ACT and Neisseria meningitidis opa proteins.
ENQNRGTHIY

SEQ ID NO: 58 - protein, 10 aa, Artificial Sequence
  Combination of Homo sapiens ACT and Neisseria meningitidis opa proteins.
TIYNTNIPGL

SEQ ID NO: 59 - protein, 10 aa, Artificial Sequence
  Combination of Homo sapiens ACT and Neisseria meningitidis opa proteins.
IYNTNIPGLT

SEQ ID NO: 60 - protein, 10 aa, Artificial Sequence
  Combination of Homo sapiens ACT and Neisseria meningitidis opa proteins.
YNTNIPGLTQ

SEQ ID NO: 61 - protein, 10 aa, Artificial Sequence
  Combination of Homo sapiens ACT and Neisseria meningitidis opa proteins.
NTNIPGLTQE

SEQ ID NO: 62 - protein, 10 aa, Artificial Sequence
  Combination of Homo sapiens ACT and Neisseria meningitidis opa proteins.
TNIPGLTQEN

SEQ ID NO: 63 - protein, 10 aa, Artificial Sequence
  Combination of Homo sapiens ACT and Neisseria meningitidis opa proteins.
NIPGLTQENQ

SEQ ID NO: 64 - protein, 10 aa, Artificial Sequence
  Combination of Homo sapiens ACT and Neisseria meningitidis opa proteins.
IPGLTQENQN

SEQ ID NO: 65 - protein, 10 aa, Artificial Sequence
  Combination of Homo sapiens ACT and Neisseria meningitidis opa proteins.
PGLTQENQNR

SEQ ID NO: 66 - protein, 10 aa, Artificial Sequence
  Combination of Homo sapiens ACT and Neisseria meningitidis opa proteins.
GLTQENQNRG

SEQ ID NO: 67 - protein, 5 aa, Homo sapiens PSA
PEDTG

SEQ ID NO: 68 - protein, 5 aa, Neisseria meningitidis opa proteins
KKTTN

SEQ ID NO: 69 - protein, 4 aa, Neisseria meningitidis opa proteins
KTTN

SEQ ID NO: 70 - protein, 10 aa, Homo sapiens PSA
HSLFHPEDTG

SEQ ID NO: 71 - protein, 15 aa, Artificial Sequence
  Xaa = Any Amino Acid (excluding cysteine, methionine, and trytophan).
CXXXXXDTGXXXXXX

SEQ ID NO: 72 - protein, 15 aa, Artificial Sequence
  Xaa = Any Amino Acid (excluding cysteine, methionine, and trytophan).
CXXXXXAQVXXXXXX

SEQ ID NO: 73 - protein, 15 aa, Artificial Sequence
  Xaa = Any Amino Acid (excluding cysteine, methionine, and trytophan).
CXXXXXIARXXXXXX

SEQ ID NO: 74 - protein, 15 aa, Artificial Sequence
  Xaa = Any Amino Acid (excluding cysteine, methionine, and trytophan).
CXXXXXLSHXXXXXX